(19) United States
(12) Patent Application Publication
(10) Pub. No.: US 2006/0088857 A1
(43) Pub. Date: Apr. 27, 2006

(54) Method for isolation of independent, parallel chemical micro-reactions using a porous filter

(76) Inventors: Said Attiya, New Haven, CT (US); Vinod B. Makhijani, Guilford, CT (US); Ming Lei, Madison, CT (US); Yi-Ju Chen, New Haven, CT (US); John Simpson, Madison, CT (US); G. Thomas Roth, Fairfield, CT (US); Chun Heen Ho, East Haven, CT (US); Yu Pengguang, Madison, CT (US)

Correspondence Address: MINTZ LEVIN COHN FERRIS GLOVSKY & POPEO, 666 THIRD AVENUE, NEW YORK, NY, 10017, US

(21) Appl. No.: 11/217,194

(22) Filed: Sep. 1, 2005

Related U.S. Application Data
(63) Continuation-in-part of application No. 11/016,942, filed on Nov. 23, 2004.
(60) Provisional application No. 60/526,160, filed on Dec. 1, 2003.

(51) Int. Cl. C12Q 1/68; C12M 1/34

(52) U.S. Cl. 435/6; 435/287.2

(57) ABSTRACT

The present invention relates to methods and apparatuses for conducting densely packed, independent chemical reactions in parallel in fluid-permeable arrays. Accordingly, this invention also focuses on the use of such arrays for applications such as DNA sequencing, most preferably pyrophosphate sequencing, and DNA amplification.

### RELATED APPLICATIONS

This application is a continuation-in-part of U.S. application Ser. No. 11/016,942 filed Nov. 23, 2004, which claims the benefit of U.S. application Ser. No. 60/526,160 filed Dec. 1, 2003, which are hereby incorporated by reference herein in their entirety.

### FIELD OF THE INVENTION

The invention describes methods and apparatuses for conducting densely packed, independent chemical reactions in parallel in a membrane reactor with mobile supports disposed thereon.

### BACKGROUND OF THE INVENTION

High throughput chemical synthesis and analysis are rapidly growing segments of technology for many areas of human endeavor, especially in the fields of material science, combinatorial chemistry, pharmaceuticals (e.g., drug synthesis, testing), and biotechnology (e.g., DNA sequencing, genotyping).

Increasing throughput in any such process requires either that individual steps of the process be performed more quickly, with emphasis placed on accelerating rate-limiting steps, or that larger numbers of independent steps be performed in parallel. One approach for conducting chemical reactions in a high throughput manner includes performing larger numbers of independent steps in parallel, and specifically conducting simultaneous, independent reactions with a multi-reactor system.

A common format for conducting parallel reactions at high throughput levels comprises two-dimensional (2-D) arrays of individual reactor vessels, such as the 96-well or 384-well microtiter plates widely used in molecular biology, cell biology, and other areas. Individual reagents, solvents, catalysts, and the like are added sequentially and/or in parallel to the appropriate wells in these arrays, and multiple reactions subsequently proceed in parallel. Individual wells may be further isolated from adjacent wells and/or from the environment by sealing means (e.g., a tight-fitting cover or adherent plastic sheet) or they may remain open. The base of the wells in such microtiter plates may or may not be provided with filters of various pore sizes.

Further increasing the number of microvessels or microreactors incorporated in such arrays has been the focus of much research. This typically involves miniaturization. For instance, the numbers of wells molded into plastic microtiter plates has steadily increased in recent years—from 96, to 384, and to 1536. Efforts to further increase the density of wells are ongoing (e.g. Matsuda and Chung, 1994; Michael et al., 1998; Taylor and Walt, 1998).

Attempts to make arrays of microwells and microvessels for use as microreactors have also been a focal point for development in the areas of microelectromechanical and micromachined systems. Researchers have applied and modified microfabrication techniques originally developed for the microelectronics industry (see Matsuda and Chung, 1994; Rai-Choudhury, 1997; Madou, 1997; Cherukuri et al., 1999; Kane et al. 1999; Anderson et al., 2000; Dannoux et al., 2000; Deng et al., 2000; Zhu et al., 2000; Ehrfeld et al., 2000).

Yet another widely applied approach for conducting miniaturized and independent reactions in parallel involves spatially localizing or immobilizing at least some of the participants in a chemical reaction on a surface. This creates large 2-D arrays of immobilized reagents. Reagents immobilized in such a manner include chemical reactants, catalysts, other reaction auxiliaries, and adsorbent molecules capable of selectively binding to complementary molecules. Microarray techniques involving immobilization on planar surfaces have been commercialized for the hybridization of oligonucleotides (e.g. by Affymetrix, Inc.) and for target drugs (e.g. by Graffinity, AB).

A major obstacle to creating microscopic, discrete centers for localized reactions is that restricting unique reactants and products to a single, desired reaction center is frequently difficult. There are two aspects to this problem. The first is that “unique” reagents—i.e., reactants and other reaction auxiliaries that are meant to differ from one reaction center to the next—must be dispensed or otherwise deployed to particular reaction centers and not to their nearby neighbors. Such “unique” reagents are to be distinguished from “common” reagents like solvents, which frequently are meant to be brought into substantial contact with all the reaction centers simultaneously and in parallel. The second aspect of this problem has to do with restricting reaction products to the vicinity of the reaction center where they were created—i.e., preventing them from traveling to other reaction centers with attendant loss of reaction fidelity.

To solve the first problem, reaction centers can consists of discrete microwells with the microvessel walls (and cover, if provided) designed to prevent fluid contact with adjacent microwells. However, delivery of reagents to individual microwells can be difficult, particularly if the wells are especially small. For example, a reactor measuring 100 μm×100 μm×100 μm has a volume of only 1 nanoliter. This can be considered a relatively large reactor volume in many types of applications. Even so, reagent addition in this case requires that sub-nanoliter volumes be dispensed with a spatial resolution and precision of at least ±50 μm. Furthermore, addition of reagents to multiple wells must be made to take place in parallel, since sequential addition of reagents to at most a few reactors at a time would be prohibitively slow. Schemes for parallel addition of reagents with such fine precision exist, but they entail some added complexity and cost.

On the other hand, the reaction centers can be brought into contact with a common fluid, e.g., such that microwells all open out onto a common volume of fluid at some point during the reaction or subsequent processing steps. However, this can cause the reaction products (and excess and/or unconverted reactants) originating in one reaction microwell or vessel to travel and contaminate adjacent reaction microwells. Such cross-contamination of reaction centers can occur (i) via bulk convection of solution comprising reactants and products from the vicinity of one well to another, (ii) by diffusion (especially over reasonably short distances) of reactant and/or product species, or (iii) by both processes occurring simultaneously.

In certain cases, the individual chemical compounds that are produced at the discrete reaction centers are themselves the desired objective of the process (e.g., as is the case in combinatorial chemistry). For such compounds, any reactant and/or product cross-contamination that may occur will reduce the yield and ultimate chemical purity of this library of discrete products. In other cases, the reaction process is conducted with the objective of obtaining information of some type, e.g., information as to the sequence or composition of DNA, RNA, or protein molecules. For these reactions, the integrity, fidelity, and signal-to-noise ratio of that information may be compromised by chemical “cross-talk” between adjacent or even distant microwells.

The issue of contamination of a reaction center or well by chemical products being generated at nearby reaction centers or microwells becomes even more problematic when reaction sites are arrayed on a 2-D surface (or wells are arranged in an essentially two-dimensional microtiter plate) over which fluid flows. In such situations, compounds produced at a surface reaction site or within a well undergo diffusive transport up and away from the surface (or out of the reaction wells), where they are subsequently swept downstream by convective transport of fluid that is passing through a flow channel in fluid communication with the top surface of the array.

### SUMMARY OF THE INVENTION

The invention encompasses novel membrane-based arrays that allow for effective trapping of mobile supports (e.g., beads or particles), fast reagent exchange, and controlled microfluidic flow. The invention further encompasses novel methods for densely packing mobile supports. This technique provides not only dense packing of reaction sites, microvessels, and reaction wells, but also provides for efficient delivery of reagents and removal of products by convective flow rather than by diffusion alone. This latter feature permits much more rapid delivery of reagents and other reaction auxiliaries. In addition, it permits faster and more complete removal of reaction products and by-products than has heretofore been possible using methods and apparatus described in the prior art. The invention pertains generally to microfluidic devices, membrane engineering, microfabrication, and convective flow methods. The present invention finds use in numerous applications including DNA sequencing, drug discovery, microimaging, microchemical reactions, substrate treatment, and high throughput screening.

One embodiment of the invention is directed to a membrane reactor comprising a porous membrane layer attached to a planar mesh array. The planar mesh comprises a plurality of openings with reactant- or reagent-carrying mobile supports of an appropriate size disposed in the openings. As an example, an appropriate size is one whereby the mobile supports are retained in the openings of the mesh. The mesh array is permeable to an aqueous fluid, such as a fluid or reagent used in sequencing but the mesh array is not permeable to the reagent- or reactant-carrying mobile supports. In a preferred embodiment, the planar mesh array is weaved from individual fibers with a spacing of less than about 100 μm center to center. In another preferred embodiment, the weaving may be made from two sets of parallel fibers that intersect at right angles. In other words, the weaving may be similar to the strings on a tennis racket at a microscopic scale.

Another embodiment of the invention is directed to a membrane reactor comprising a porous membrane and a planar array which is fabricated above the top surface of the membrane. The planar array comprises a plurality of wells for trapping mobile supports. The pores in the membrane are sufficiently sized such that the membrane is permeable to fluids but impermeable to the mobile supports. Each well in the array has an opening of less than about 40 μm. That is, for an array with a well size of 40 μm, each mobile support should be somewhat smaller than 40 μm in diameter. In a preferred embodiment, the mobile supports are 2-3 μm smaller than the well width. This relationship between mobile support size and well size also ensures that only one or fewer mobile supports are immobilized to a well.

In a preferred embodiment, a plurality of wells in the planar fabricated array comprise one or fewer mobile supports. The array is in direct or indirect contact with the top surface of the porous supporting membrane. The array is contacted with a fluidic stream (e.g., vertical or near-vertical) to maintain the mobile supports in the wells by convective force. The fluidic stream also carries reagents for reacting with chemical groups on the mobile supports. Micropores in the membrane allow flow-through and provide flow resistance for the membrane reactor. The wells comprise sidewalls and bottoms to reduce physical and chemical cross-talk between the wells. Opaque sidewalls in the wells prevent optical crosstalk, while opaque bottoms prevent optical bleeding between the wells. The sidewalls and bottoms for the wells also concentrate the optical signal generated by the mobile support. The signals generated by reactions in the wells are detected by optical or electronic means.

Another embodiment of the invention is directed to a method of loading a membrane reactor with mobile supports. In the method, a membrane that is substantially permeable to a fluid but substantially impermeable to a population of mobile supports is provided. A planar array comprising wells is positioned above this membrane. A fluid comprising a suspension of said population of mobile supports is introduced onto the surface of the array. The mobile supports may be linked to a sample (e.g., nucleic acid or peptide) or they may be unlinked. The mobile supports are settled onto the wells of the array, preferably using a pump or negative pressure or suction. Settling may be performed, for example by allowing the mobile supports to slowly settle out of solution under gravity. Another method of settling may involve centrifugation. In a preferred method, the fluid is drawn through the array and membrane. Since the mobile supports are larger than the pores of the membrane, they are trapped (loaded) in the wells of the array as the fluid is drawn through.

Another embodiment of the invention is directed to a method of identifying a base at a target position (e.g., sequencing) in one or more sample nucleic acid, preferably DNA. Preferably, the sequencing reaction is a pyrophosphate sequencing reaction. In one aspect of the method, the sample DNA is immobilized on a mobile support on the membrane reactor. An extension primer is used to hybridize to the sample DNA immediately adjacent to the target position. The extension primer is subjected to a polymerase reaction in the presence of a deoxynucleotide or dideoxynucleotide so that the deoxynucleotide or dideoxynucleotide will only become incorporated and release pyrophosphate (PPᵢ) if it is complementary to the base in the target position. Any release of PPᵢ is detected enzymatically, such as, for example, by detecting a light emission generated by an enzyme in response to the presence of PPᵢ. In various aspects, the light emissions are generated directly or through a chemical pathway involving additional chemical steps or amplification steps.

In one preferred embodiment, the sequencing reagents, including the deoxynucleotides or dideoxynucleotides, are contacted to the nucleic acid by a flow of reagent that is normal (i.e., orthogonal, perpendicular) to the plane of the membrane reactor. Because the flow is normal to the plane of the mobile supports, each fluid stream will only contact one mobile support or one species of nucleic acid before it is disposed into a waste container. Such reagent flow is useful for reducing or eliminating cross contamination between wells in the array. In this method, the deoxynucleotides or dideoxynucleotides are added successively to the sample-primer mixture and subjected to the polymerase reaction to indicate which deoxynucleotide or dideoxynucleotide is incorporated.

Another embodiment of the invention is directed to a microimaging system for imaging a light emission (e.g., from a pyrophosphate sequencing reaction) from a membrane reactor. The system comprises one or more lens groups. The first lens group is the front lens group which is positioned closer to the light source to be detected to collect the light that is emitted. The second lens group is the rear lens group which is positioned closer to the light detector such as a CCD detection device to image the light onto the detector. In a preferred embodiment, the lens groups comprise 50 mm lenses with an aperture larger than or equal to 2.8 (e.g., 2.0, 1.8, 1.4, 1.0, etc.). It should be noted that the larger apertures are expressed by a smaller aperture value so that, for example, an aperture of 1 is larger than an aperture of 2.

Another embodiment of the invention is directed to a sequencing cartridge. The cartridge comprises a flow chamber for enclosing an above described membrane reactor. A membrane supporting structure inside the flow chamber separates the flow chamber into two subchambers. The first subchamber comprises the membrane reactor and also comprises an inlet and a first outlet for controlling a fluid flow tangential to the membrane reactor. The first subchamber also comprises a window, covered with a transparent material such as glass or crystal, to allow the optical examination of the membrane reactor. The second subchamber without the membrane reactor comprises a second outlet allowing fluid to flow normally (i.e., orthogonally) from the inlet, through the membrane reactor, and out through the second outlet. In this manner, both the tangential and normal flow of reagent through the membrane reactor may be regulated.

Another embodiment of the invention is directed to a method of amplifying a sample nucleic acid on a mobile support and then loading the mobile support on a membrane reactor. In this method, one or more nucleic acid templates to be amplified are individually attached to separate mobile supports to form a population of nucleic acid template-carrying supports. The template-carrying supports are suspended in an amplification reaction solution comprising reagents necessary to perform nucleic acid amplification. An emulsion is formed to encapsulate the plurality of said template-carrying supports with PCR reaction solution to form a plurality of microreactors (see, e.g., U.S. application Ser. No. 60/476,504, filed Jun. 6, 2003; U.S. application Ser. No. 10/767,899, filed Jan. 28, 2004, and PCT/US04/02484 filed Jan. 28, 2004, which are hereby incorporated by reference in its entirety). One or more nucleic acid templates in fluidic isolation from each other are then amplified to form multiple copies of nucleic acid templates. The amplified nucleic acid templates, still in fluidic isolation, are attached to the mobile supports. The mobile supports are loaded into the membrane reactor.

Another embodiment of the invention is directed to a method of producing a membrane reactor by providing one or more nucleic acid templates to be amplified, wherein a plurality of nucleic acid templates are individually attached to separate mobile supports to form a population of nucleic acid template-carrying supports. The mobile supports are loaded onto the membrane reactor. After loading, the template-carrying supports are contacted to an amplification reaction solution comprising reagents necessary to perform nucleic acid amplification. Then, the nucleic acid template is amplified in fluidic isolation from other templates to form amplified nucleic acid. Fluidic isolation may be achieved, for example, by removing most fluids from the membrane reactor and allowing amplification on the fluids that is still in contact with the mobile supports. Furthermore, oil may be added to the membrane reactor to prevent evaporation during amplification, and then removed by organic solvents such as hexane.

For purposes of this patent specification, the selective binding of one molecule to another—whether reversible or irreversible—will be referred to as a reaction process, and molecules capable of binding in such a manner will be referred to as reactants. Immobilization may be arranged to take place on any number of substrates, including planar surfaces and/or high surface area and sometimes porous support media such as beads or gels.

### BRIEF DESCRIPTION OF THE DRAWINGS

FIG. 1 represents an integral or physical composite of a microchannel array and a porous membrane barrier forming a membrane reactor. The flow of fluid through the membrane reactor carries reaction participants along with the fluid.

FIG. 2 shows a schematic of one version of the experimental set-up for the convective flow sequencing apparatus described herein.

FIG. 3 shows a membrane reactor comprising a nylon mesh membrane useful for trapping reagent- or reactant-carrying mobile supports and one embodiment of the mobile supports. Shown here are Sepharose beads.

FIGS. 4A-4B show the size of the Sepharose beads relative to the membrane pores. In FIG. 4A, the beads are shown to be swollen in liquid. In contrast, FIG. 4B shows how the beads are shrunken when dry.

FIG. 5A represents a membrane holder with a circular optical window; a flow chamber with an inlet port and a first outlet port; a nylon membrane with sepharose beads; a fine pore nylon membrane; a membrane support structure with 1.02 mm holes, 1.35 pitch; and a funnel-shaped collector with a second outlet port. FIG. 5B represents a support structure.

FIG. 6A shows a membrane support structure with a 50 mm capillary plate with 10 μm holes and a 12 μm pitch for supporting the membrane reactor array. FIG. 6B shows 5× magnification of the membrane. FIG. 6C shows 40× magnification of the membrane.

FIG. 7 shows a schematic of a pyrophosphate-based sequencing method with photon detection.

FIG. 8 shows an automated convective sequencing apparatus.

FIG. 9 shows a sequencing pyrogram indicating the results of sequence analysis. The pyrogram sequence (top; SEQ ID NO:5) and signal intensity sequence (bottom; SEQ ID NO:10) are shown.

FIGS. 10A-10B show a sequencing graph. FIG. 10A shows the results for the negative control (no template added), where no sequence was detected. FIG. 10B shows the results for the template, where the correct sequence was detected (SEQ ID NO: 11).

FIG. 11A shows a schematic for the coupling of amine-primer and amine-biotin to NHS activated sepharose beads. FIG. 11B shows a schematic for the addition of biotinylated sulfurylase and luciferase.

FIG. 12 represents a side-view of a single well of the planar array layer of the membrane reactor. The well encloses one bead and comprises opaque sidewalls and an opaque or reflective bottom. The bead is positioned over a porous membrane layer which is permeable to a microfluidic flow path.

FIG. 13A represents a side-view of the porous membrane layer. FIG. 13B depicts a top view of the porous membrane layer. FIG. 13C depicts a porous membrane layer being positioned with forceps.

FIGS. 14A-14B show schematic steps for the construction of a membrane reactor. Features are not drawn to scale. FIG. 14A depicts metal deposition on the porous membrane layer. FIG. 14B depicts photoresist coating on the porous membrane layer. FIG. 14C depicts the photolithography process. FIG. 14D depicts development of the photoresist. FIG. 14E depicts the electroplating process. FIG. 14F depicts removal of the photoresist. FIG. 14G depicts optional gold, chromium, or titanium etching. FIG. 14H depicts bead loading and trapping. FIG. 14I depicts the fluid convention process.

FIG. 15 represents a top view of two wells of a planar array layer positioned over a porous membrane layer. Features are not shown to scale.

FIGS. 16A-16D represent various well configurations.

FIG. 17 shows the optical paths in a well in a planar array layer. The sidewalls of the well concentrate light and reduce optical bleeding. The bottom of the well reduces light scattering and increases reflection.

FIGS. 18A-18B shows an example of a mold plating system for producing electroplated microstructures (see, e.g., J. B. Lee, University of Texas). FIG. 18A depicts the mold. FIG. 18B depicts the electroplated microstructure.

FIG. 19A represents a membrane reactor with sidewalls and structural support beams. A post structure is shown with a square cross-section, arranged in hexagonal shape. FIGS. 19B-19C depict different membrane configurations. In FIG. 19B, three types of post structures are shown. The cross-sections of the posts are in diamond or circular shapes, while the array is square or hexagonal. In FIG. 19C, a reversed structure is shown in which wells are formed on substrate surface or in a built-on top layer.

FIG. 20 represents a membrane reactor in contact with a CCD imaging screen.

FIG. 21A represents a cross-section of a membrane reactor showing the planar array layer and porous membrane layer. FIG. 21B shows schematic steps for the construction of a membrane reactor including metal deposition (e.g., chromium) on the substrate, photolithography, electroplating, and removal of the photoresist and chromium layers.

FIG. 22 represents various gaskets, holders, rivets, and supports for the planar array and the porous membrane for the membrane reactor.

FIG. 23A depicts a membrane reactor comprising sloped wells and beads. FIG. 23B represents a cross-section of the sloped wells and beads.

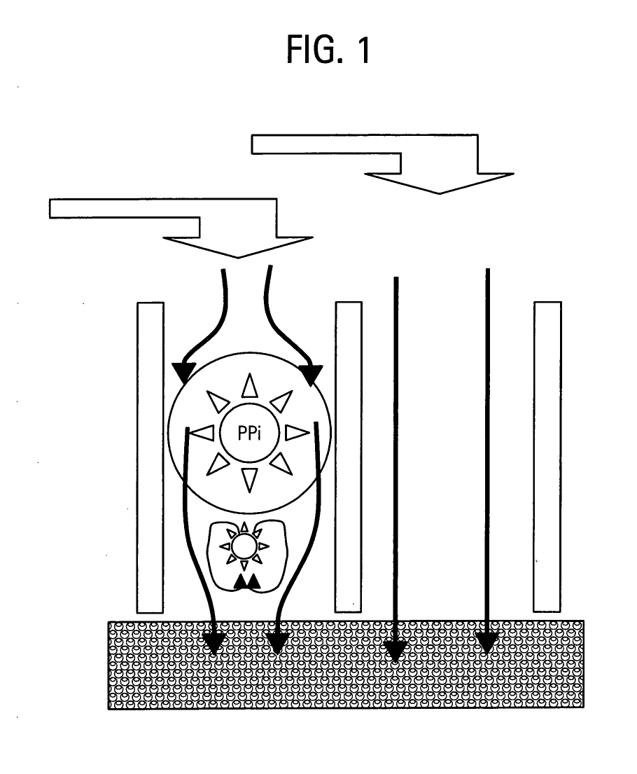

### DETAILED DESCRIPTION OF THE INVENTION

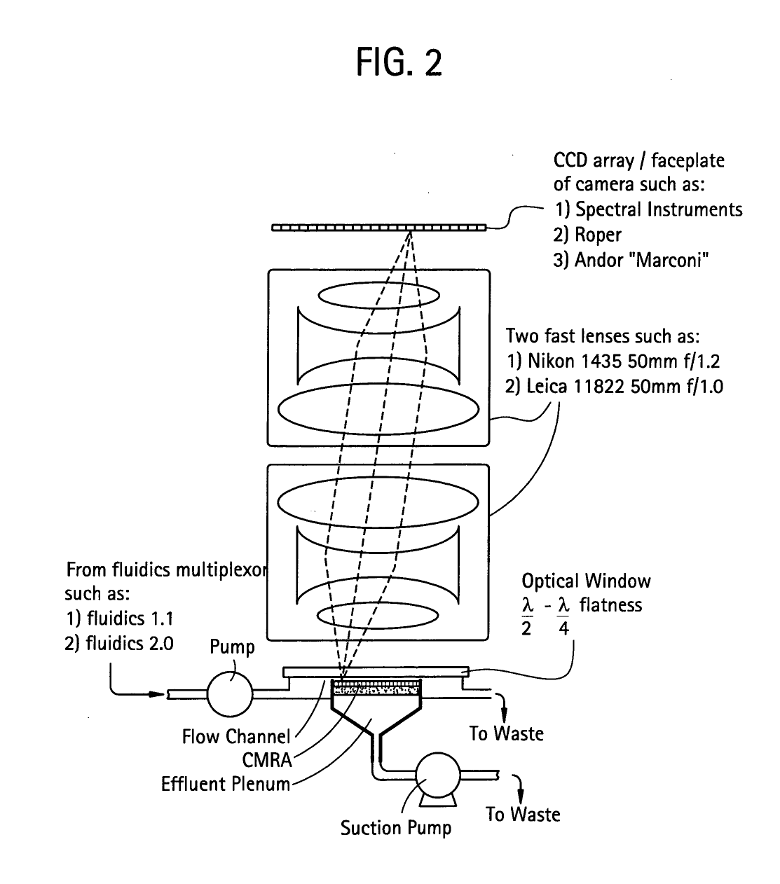

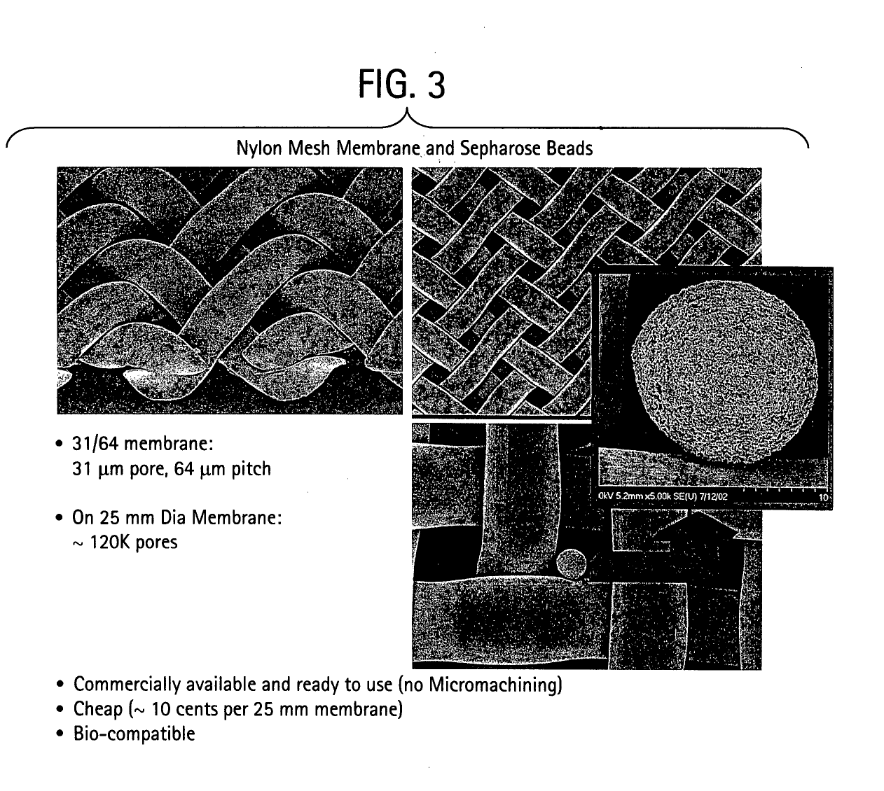

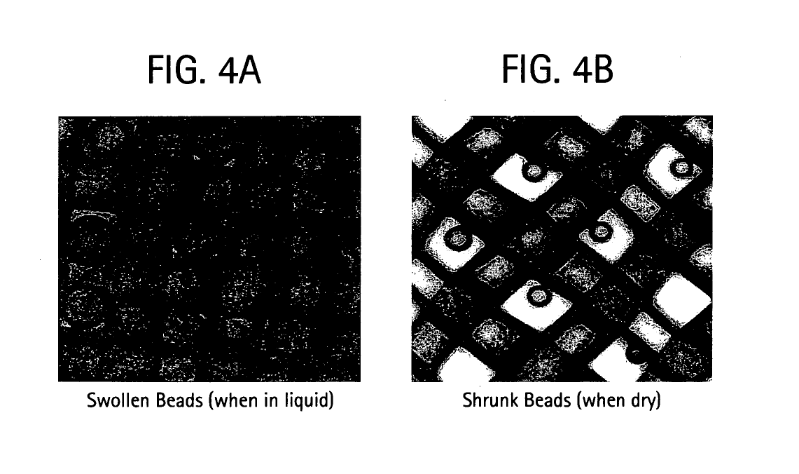

For the purposes of this patent application, the membrane reactor is a general term that describes both the confined membrane reactor array (CMRA) and the unconfined membrane reactor array (UMRA) as described by U.S. application Ser. No. 10/191,438 filed Jul. 8, 2002, the entire contents of which are incorporated herein by reference. Methods and apparatuses are described here for providing a dense array of discrete reaction sites, microreactor vessels, and/or microwells (see FIG. 2) and for charging such microreactors with reaction participants by affecting a convective flow of fluid normal to the plane of and through the array of reaction sites or microvessels. The convective flow or delivery of reactants includes both the delivery of sequencing reactants (e.g., dNTPs) towards the reaction site, and convective removal of excess reactants away from the site. Fluid flow sweeps the sequencing reaction products (e.g., pyrophosphate (PPᵢ), ATP) through the reaction region in a normal direction thus countering back-diffusion and resultant contamination.

Reaction participants that may be charged, concentrated, and contained within said reaction sites or microreactor vessels by methods of the present invention include high-molecular-weight reactants, catalysts, and other reagents and reaction auxiliaries. In the context of oligonucleotide sequencing and DNA/RNA analysis, such high-molecular-weight reactants include, for example, oligonucleotides, longer DNA/RNA fragments, and constructs thereof. These reactants may be free and unattached (if their molecular weight is sufficient to permit them to be contained by the method of the present invention), or they may be covalently bound to or otherwise associated with, e.g., high-molecular-weight polymers, high-surface-area mobile supports, or gels, or other supports known in the art.

Examples of reaction catalysts that may be similarly delivered to and localized within said reaction sites or microvessels include enzymes, which may or may not be associated with or bound to solid phase supports such as porous or non-porous mobile supports (e.g., beads or particles). As another example, enzymes such as polymerase may be attached to supports as a reagent. In addition, additional polymerase may be delivered during a reaction to replenish, or supplement the bound polymerase. In this example, a reagent (e.g., polymerase) is both free and unattached and covalently bound or associated with the support. Any reagent or reactant of this invention may be both free and bound as described herein.

The present invention also includes a means for efficiently supplying relatively lower-molecular-weight reagents and reactants to said discrete reaction sites or microreactor vessels. Also included are means for efficiently removing unconverted reactants and reaction products from said reaction sites or microvessels. More particularly, efficient reagent delivery and product removal are accomplished in the present invention by arranging for at least some convective flow of solution to take place in a direction normal to the plane of the substantially two-dimensional array of reaction sites or microreactor vessels. This flow can lead past or through the discrete sites or microvessels, respectively, where chemical reaction takes place. In this instance, reactants and products will not necessarily be retained or concentrated at the reaction sites or within the reaction microvessels or microwells; indeed, it may be desired that certain reaction products be rapidly swept away from and/or out of said reaction sites or microvessels.

This invention also minimizes the amount of contamination among neighboring reaction sites or “blow-by” which typically occurs in diffusive sequencing. In diffusive sequencing, reaction products from an upstream site have multiple chances to contaminate downstream sites. This contamination is a cumulative effect that may worsen if there are a large number of DNA fragments and multiplets in the upstream reaction sites. In the present invention, the possibility of blow-by has been minimized such that any possible contamination is not cumulative. Specifically, a fluid sheath is formed over each reaction site such that flow downwards relative to the flow laterally is sufficient to prevent blow-by or contamination of neighboring reaction sites. Also, each of the mobile supports is washed independently by downward flow of wash solution so that the washing of each reaction site (and any mobile support disposed therein) is independent of washing of neighboring reaction sites during the washing step.

In addition to including means for providing a controlled convective flux of fluid normal to and across the substantially planar array of reaction sites or microreactors, the present invention also includes permselective, porous filter means capable of discriminating between large (i.e., high-molecular-weight) and small (i.e., low-molecular-weight) reaction participants. This filter means is capable of selectively capturing or retaining certain reaction participants while permitting others to be flushed through and/or out the bottom of the microreactor array. By proper selection of the porous filter and the judicious choice of convective flux rates, considerable control over the location, concentration, and fate of reaction participants can be realized.

Membrane Reactors

In a preferred embodiment, the apparatus of the present invention consists of an array of microreactor elements comprised of at least two functional elements that may take various physical or structural forms. These include: (i) a planar array layer comprised of an array of microchannels or microwells and on average no more than one mobile support (e.g., reagent- or reactant-carrying mobile support) disposed therein, and (ii) a porous membrane layer comprising, e.g., a porous film or membrane in the form of a sheet or thin layer. These two elements are arranged next to and in close proximity or contact with one another, with the plane of the microchannel/microvessel element parallel to the plane of the porous membrane element. In other words, the planar array layer and the porous membrane layer may be in contact with each other to form one sheet with two layers.

As referred to herein, the side of this composite structure containing the microchannel or microvessel array will be referred to hereinafter as the “top”, while the side defined by the porous membrane will be referred to as the “bottom” of the structure. In a preferred embodiment, contact between the planar array and the porous membrane may be tight, in which case a fluid cannot travel from the bottom side of one microchannel into another microchannel without entering and exiting the porous membrane element. In another embodiment, the contact may also be loose, in which case some fluid may travel from one microchannel to another on the bottom of the planar array layer without passage through the porous membrane element. In practice, the flow of fluids in a direction normal to the plane of the membrane reactor would prevent significant cross contamination between microchannels even if the contacts were loose.

In its various advantages, the membrane reactor of the invention allows increased trapping efficiency for mobile supports, high density deposition of mobile supports, and loading of one or fewer mobile supports per well. The membrane reactor is amenable for use with automatic or semi-automatic deposition processes for mobile supports. The membrane reactor allows variations in pitch and density of the planar array and adjustment of flow resistance, which can be used to improve microfluidic flow distribution around the mobile supports. The membrane reactor optimizes stability and flatness to enhance imaging quality and improve high throughput screening. The membrane reactor is easily assembled by batch fabrication processes, which are cost effective for small or large scale productions. As important advantages, the membrane reactor is designed to reduce or eliminate optical or chemical cross-talk, and blow-by from reagents in adjacent wells. The membrane reactor also allows tracking of locations for individual mobile supports location.

The membrane reactor will be discussed in more detail following the discussions of each of its component elements.

Planar Array Layer

The planar array layer comprises individual wells (also called microchannels, microvessels, reaction chambers). Each well consists of a single microchannel. The planar array layer comprises a plurality of wells, with the longitudinal axes of said wells being arranged in a substantially parallel manner, and with the downstream ends of said channels being in functional contact with a porous membrane (described below). The aspect ratio of the microchannels (i.e., their height- or length-to-diameter ratio) may be small or large, and their cross-section may take any of a number of shapes (e.g., circular, rectangular, hexagonal, etc.). As discussed further below, it is not at all essential that the microchannel walls be continuous or regular. It is preferred that the effective well size of the array layer is comparable to or slightly larger than the diameter of the mobile supports that one desires to retain.

In one embodiment, the array layer typically comprises at least 10,000 wells, at least 50,000 wells, at least 100,000 wells, or at least 250,00 wells, and in one preferred embodiment, between about 100,000 and 1,000,000 wells, and in another preferred embodiment, between about 250,000 and 750,000 wells. Most preferably, the array layer comprises at least about 100, 100-1000, 1000-10,000, 10,000-20,000, 20,000-30,000, or 32,000 wells per mm². The array layer is typically constructed to have wells with a center-to-center (c-t-c) spacing less than 100 μm, preferably about 5 to 200 μm, preferably about 10 to 150 μm, even more preferably about 25 to 100 μm, and most preferably about 50 to 78 μm. In five preferred embodiments, the center to center (c-t-c) spacing is less than or equal to about 58 μm, 64 μm, 68 μm, 70 μm, or 100 μm, respectively. Most preferably, the c-t-c spacing is less than or equal to about 100 μm, 32 μm, 10 μm, 7 μm, 5.7 μm, or 5.6 μm.

In one embodiment, we contemplate that each reaction chamber in the array layer has a well width in at least one dimension of between about 5 μm and 200 μm, preferably between about 10 μm and 150 μm, more preferably between about 15 μm and 100 μm, most preferably between about 20 μm and 35 μm. In one embodiment, the reaction chamber can be square and can have the above cited dimensions (or can be rectangular with those dimensions along one linear dimension of the rectangle). For substantially square wells, the average size can include, e.g., about 15 to 100 μm in width, or preferably, about 20 to 35 μm in width. In four preferred embodiments, the reaction chamber is square with well widths of about 25 μm, 28 μm, 30 μm, or 31 μm, respectively.

In four preferred embodiments, the array layer is selected from a nylon membrane with: (1) a c-t-c spacing of about 64 μm and a 31 μm well width; (2) a c-t-c spacing of about 58 μm and a 25 μm well width; (3) a c-t-c spacing of about 70 μm and a 30 μm well width; and (4) a c-t-c spacing of about 68 μm and a 28 μm well width. A preferred well width is determined by bead size. For example, if a bead is about 25 μm in a diameter, then a preferred well diameter can be about 30 μm. As other examples, the bead diameter can be about 80%, 83%, 85%, 87%, 90%, 93%, or 95% of the well diameter.

The mobile supports of the invention can comprise one or more suitable materials, including glass, silica, dextrans, ceramics, metals, or plastics. Some examples of preferred materials include Sephadex, Sepharose, agarose, polysulfone, polypropylene, polyethylene, polycarbonate, polyethyleneterephthalate, polyethersulfone, polystyrene, polytetrafluoroethylene, carboxymethyl cellulose, cellulose acetate, cellulose butyrate, polyvinylidene fluoride, acrylonitrile PVC copolymer, polyaminemethylvinylether maleic acid copolymer, polystyrene/acrylonitrile copolymer, and any combination thereof. Preferred beads include sizes of about 25 to 28 μm in diameter. For higher density loading, bead size can be, e.g., about 15 μm in a diameter.

It is advantageous to deposit or settle a particular reactant molecule (e.g., an oligonucleotide or construct thereof) at discrete sites on the surface of a membrane reactor, for example, for pyrophosphate sequencing. This may be accomplished by immobilizing said reactants on particulate or colloidal supports (e.g., beads, particles), suspending the supports in a fluid, and then depositing or settling these onto the membrane reactor surface by drawing the fluid through the membrane reactor. One method for depositing a mobile support is to place a fluid suspension of mobile supports on a membrane reactor and allow gravity to deposit the mobile supports into the individual wells. This process that can be accelerated by vibration or centrifugation. It is recognized that there may be infrequent times where more than one bead is disposed in a well but this is not preferred.

In one preferred embodiment, the mobile supports in the wells reduce the size of the wells but do not eliminate the opening in the wells. In one aspect, the mobile supports are spherical while the wells are square (see Examples). The deposition of a round mobile support in a square well would still allow a flow of fluid through the well. In another embodiment, the mobile supports and the wells have irregular shapes that deviate, slightly or grossly, from a perfect sphere and a perfect circle. The deposition of an imperfect spherical mobile support onto an imperfect circular well would not completely block the well. For example, in a preferred embodiment, the planar array layer is a fabricated or micromachined to comprise round or square wells.

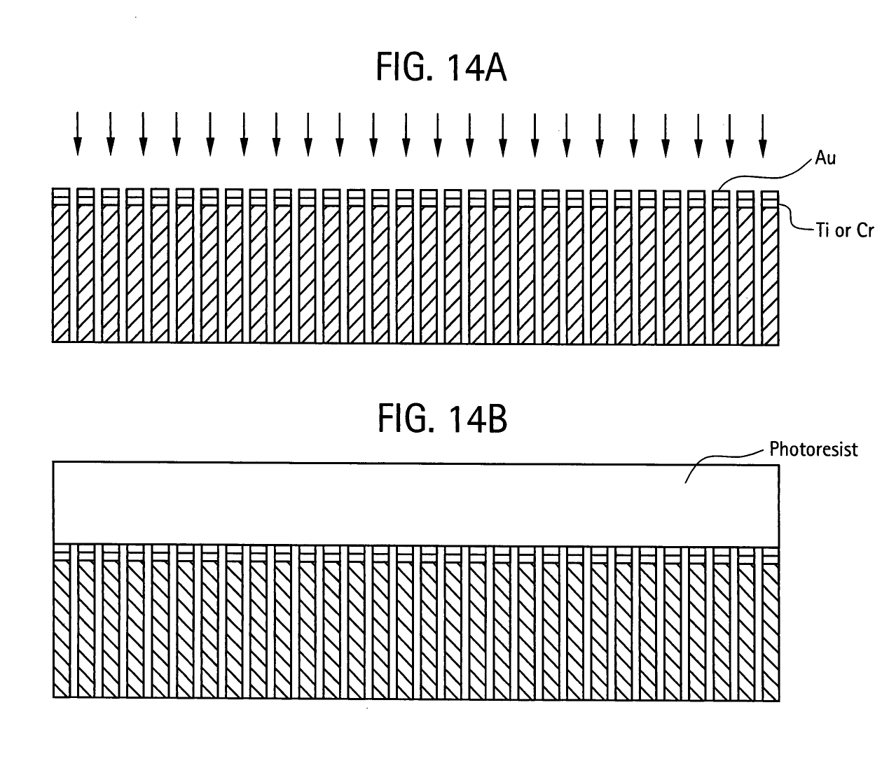

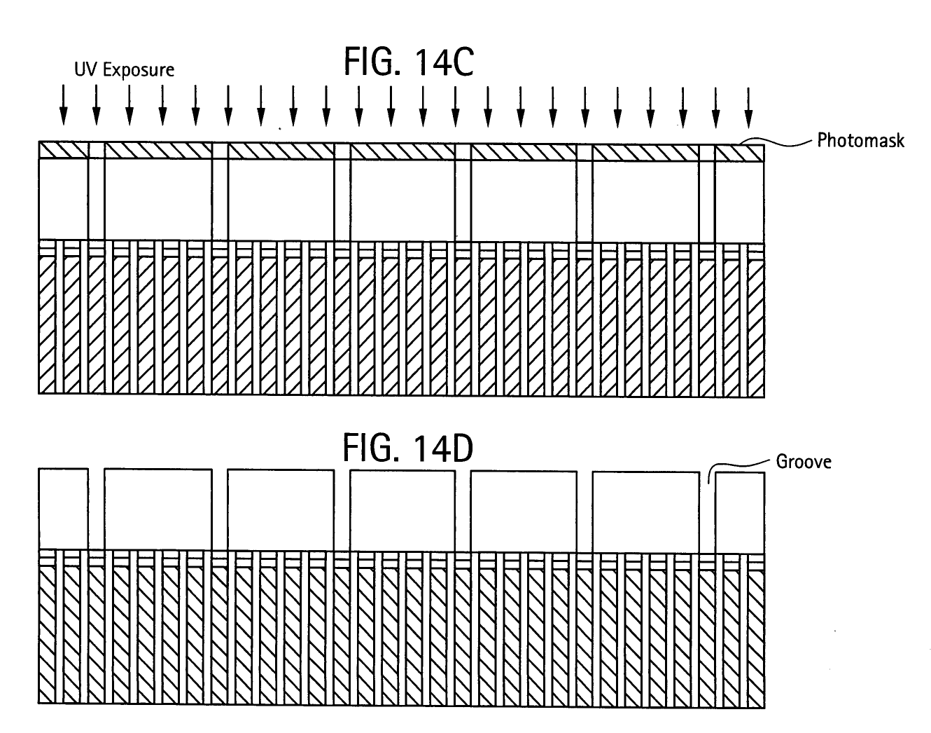

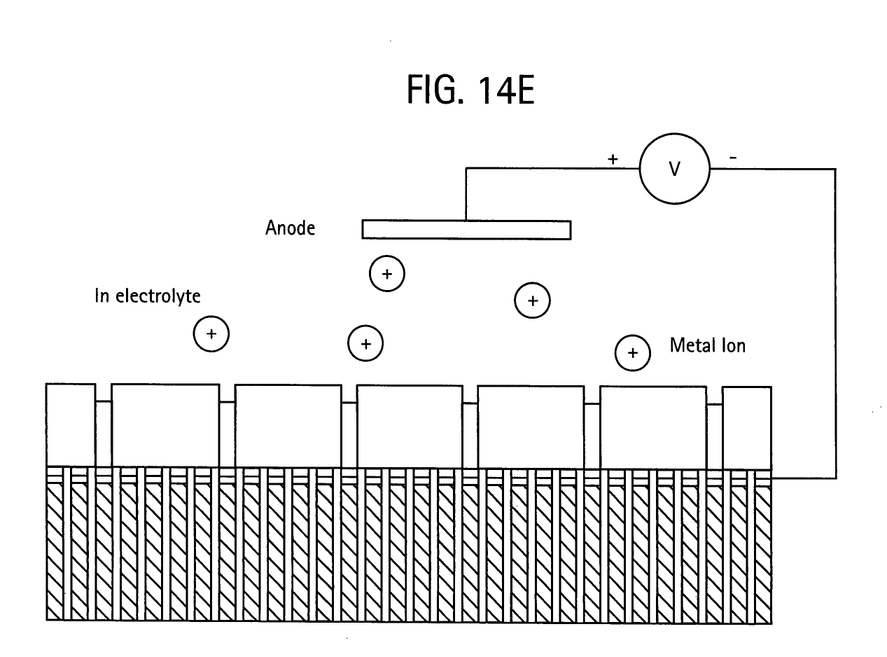

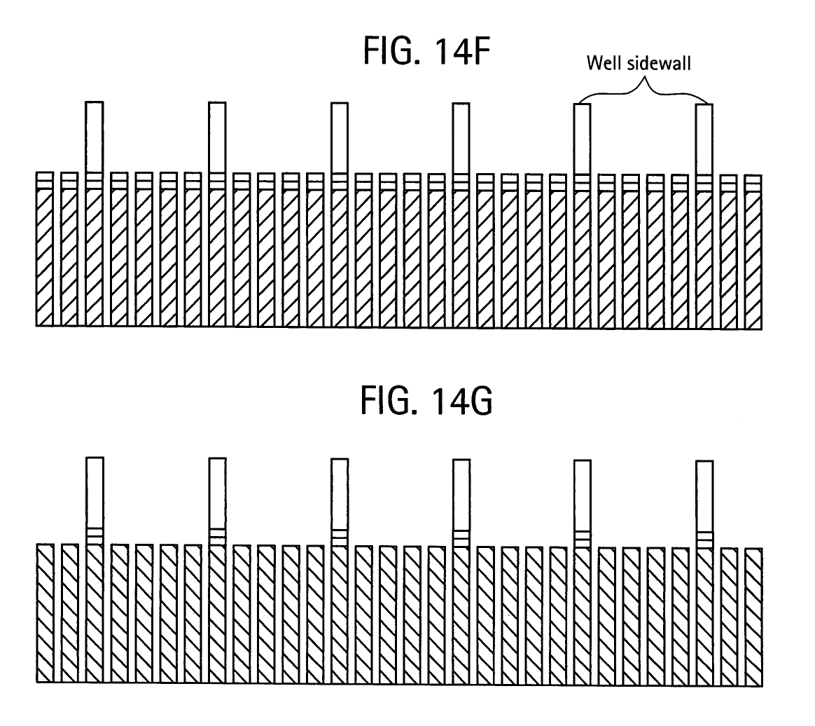

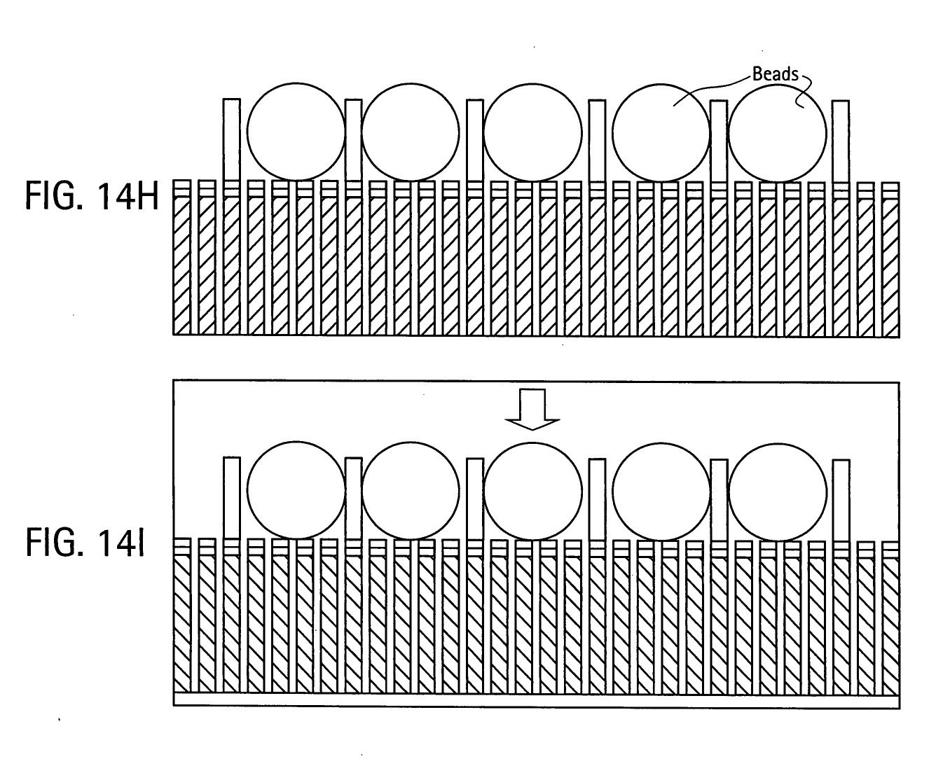

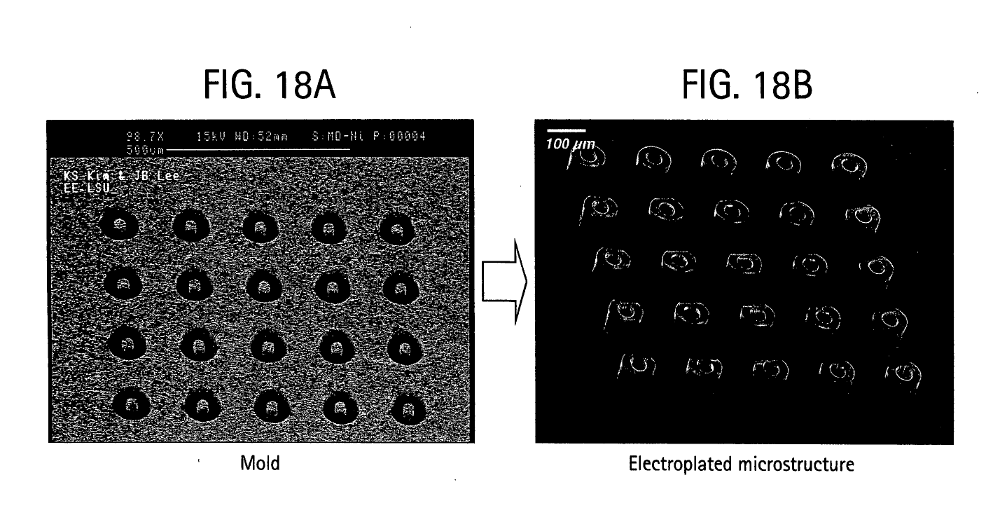

In various aspects, the planar array can be constructed on the surface of a substrate using photolithography and electroplating techniques (e.g., FIGS. 14A-14I). Alternatively, the planar array can be produced by micromolding (e.g., FIGS. 18A-18B). The planar array can also be built on the surface of a substrate, such as a fiber bundle plate, wafer, film, or sheet. For built-on structures, the flat and solid areas of the substrate are used as a foundation for building. For photolithography, the substrate is coated by metal deposition (e.g., gold and titanium or chromium) with photoresist and the pattern of the structure is generated by a photomask. The metal layer can be, e.g., about 0.05 μm, 0.07 μm, 0.1 μm, or 0.15 μm in thickness. The photoresist layer can be, e.g., about 25 μm, 35 μm, 50 μm, or 57 μm in thickness.

The pattern is transferred from the photomask onto the photoresist coating using UV exposure. The photoresist coating can comprise SU-8 film or other photosensitive materials. After photolithography process, the substrate is submerged in an electrolyte solution for electroplating. Metal deposition occurs only in exposed grooves. The thickness of plated structure is controlled by time and current density or voltage. Following the completion of electroplating, the photoresist layer is removed to produce sidewalls. The sidewalls form the wells of the planar array layer.

The pitch of the wells (i.e., the distance between the centre of one well and the next) and array patterns can be designed according to need. It is possible to vary the total density of wells per area unit, and produce any desired array pattern, for example, square, hexagonal or redial patterns. Square and hexagonal shapes are most efficient for use of space and trapping beads. Usually, posts or well sizes inside an array are uniform in size. However, it is possible to build posts or wells in variety of sizes in order to produce specific functions. For example, one or more wells in an hexagonal array (e.g., center well) can be made smaller than others or completely filled (i.e., include no cavity). This is particular useful for image recognition. The configuration of wells can also be varied depending on requirements of microfluidic flowing, resistance criteria, and special flow distribution requirements. To produce minimal scatter of light from the surface of the membrane, additional metal thin film can be deposited onto the surface to produce a near black top layer. This thin layer of metal can be blasted onto the surface by electroplating, thermal evaporation, or sputtering processes. Metal thin film can remain at the bottom of the wells or be removed by additional etching. In preferred aspects, the sidewalls form wells that are slightly wider than the mobile supports. This allows for trapping single mobile supports in the wells.

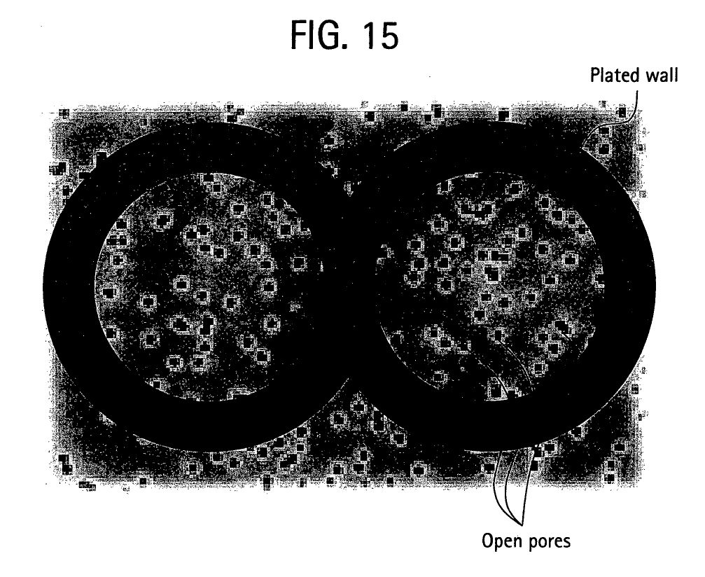

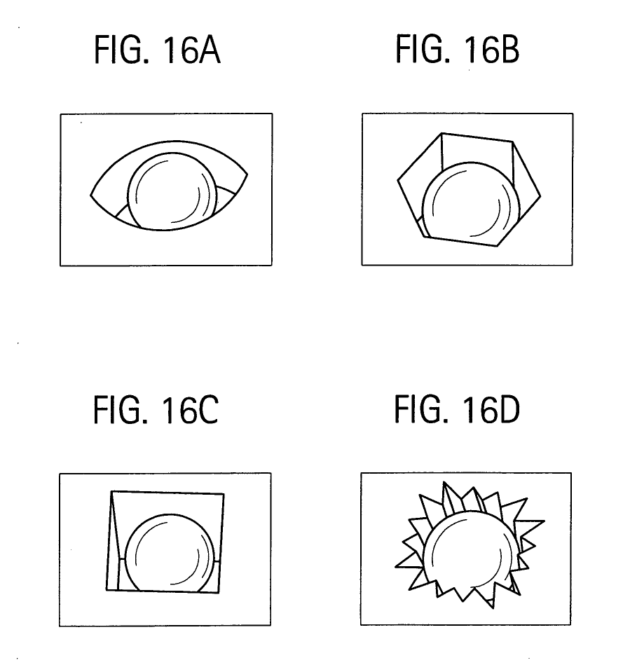

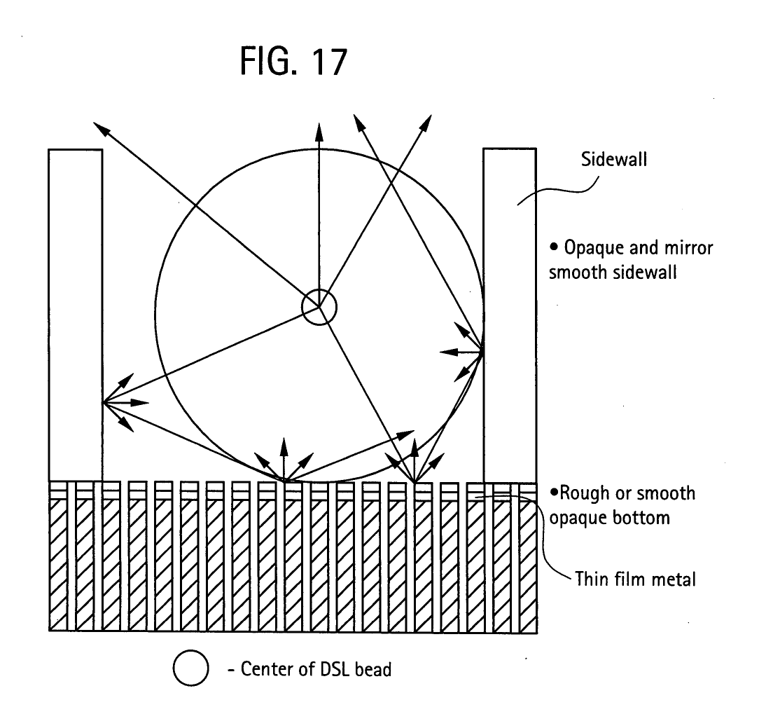

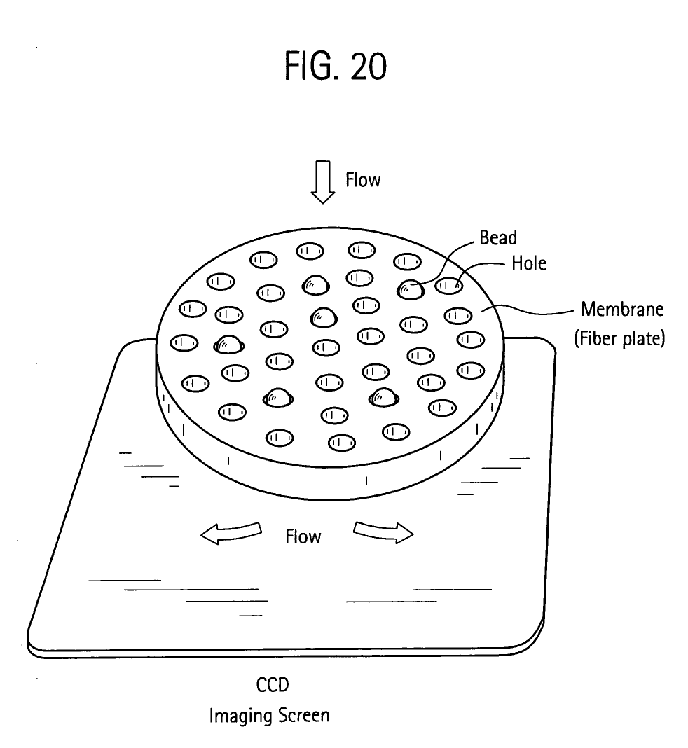

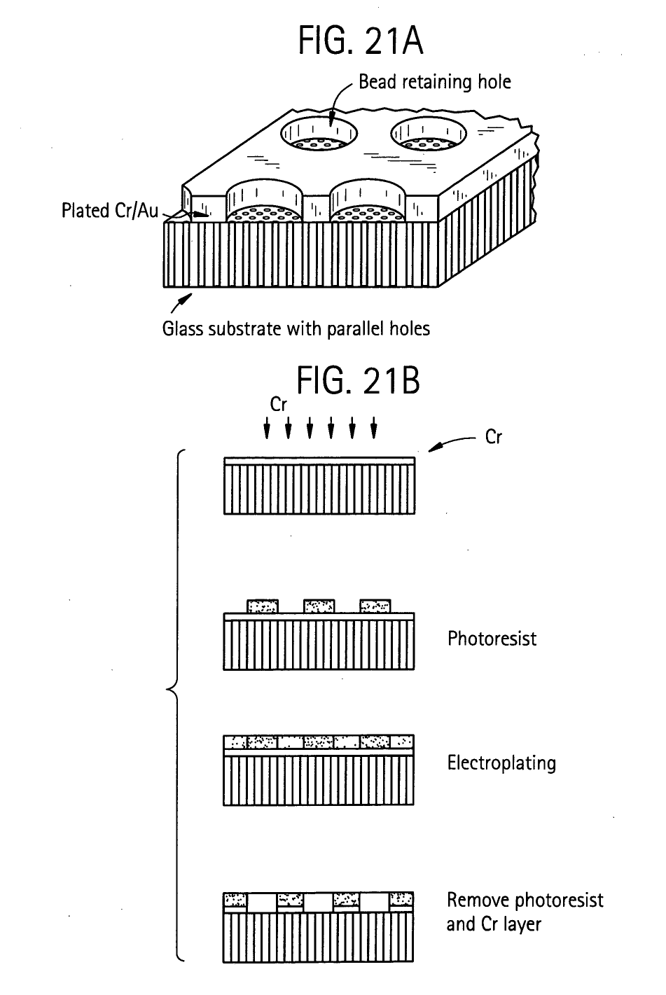

The wells can include opaque sidewalls and bottoms to prevent optical crosstalk, e.g., between mobile supports and between wells (see, e.g., FIG. 17). The heights of the sidewalls are controlled during the electroplating process. Preferably, the sidewalls are higher than the mobile supports (e.g., the walls are higher than the diameter of the beads). Most preferably, the sidewalls are only slightly higher than the mobile support. Sidewalls that are substantially higher than the mobile supports will allow multiple supports to load in each well. The wells can be formed in patterns, such as regular arrays, or in an irregular distribution. The wells can comprise one or more shapes, e.g., substantially round, square, oval, rectangular, hexagonal, crescent, and/or star-shaped wells (e.g., FIGS. 15, 16A-16D, 19, 20, and 21A). The pitch of the well can be varied, for example, at least 15 μm, at least 35 μm, or at least 50 μm pitch. Reference marks (e.g., anchors) can be integrated in these patterns and used to locate the wells.

The sidewalls can be oriented at any angle on the surface of substrate. Angles can be varied on different membranes or on different areas of one membrane. Beams can be placed among plated structure to reinforce the membrane mechanical strength (see below). The sidewalls can be composed of single metals, alloys, metal-plated materials and/or laminated layers, and can include porous, black, matte, shiny, reflective, or mirrored surfaces. Layered metals can be introduced with different colors, different surface morphologies, and different composites. The sidewall can also be coated with one or more additional layers of materials, such as thin film metal, insulation coating, for example, Teflon and metal oxide for improving optical properties.

Contemplated for use with the invention are commercially available materials. Materials for the planar array layer of invention include nylon or nitrocellulose membranes and precision woven open mesh fabrics, especially monofilament open mesh fabrics, such as those available from Sefar, Inc. (Ruschlikon, Switzerland). Non-limiting examples of such fabrics include Sefar Nitex (PA 6.6), e.g., Cat. # 03-25/14, 03-28/17, 03-30/18, 03-30/20, and 03-35/16. Other materials for the planar array layer include woven nylon net filters such as those available from Millipore (Bedford, Mass.), including, but not limited to, Cat. # NY41 025 00, NY41 047 00, NY41 090 00, and NY41 000 10. For photolithography, negative acting, epoxy-type photoresists are preferred, e.g., SU-8, SU-8 5, SU-8 50, SU 8-100, SU-8 2000, EPONS resin SU-8, NANO SU-8, MCC SU8-10, BCB, and NR9-8000. For electroplating, useful metals include, but are not limited to, copper (Cu), gold (Au), iron (Fe), nickel (Ni), silver (Ag), zinc (Zn), cadmium (Cd), tin (Ti), lead (Pb), antimony (Sb), cobalt (Co), and any alloys thereof, e.g., Ti/Pb. Preferred metals for this aspect include nickel, chromium, and silver, as well as combinations comprising silver and chromium or silver and nickel. Gold as the top layer of coating is also preferred.

Porous Membrane Layer

Membrane reactors without a porous high flow resistance membrane element may suffer from non-uniform flow of reagents. In the absence of a porous high flow resistance membrane, a well with a mobile support would have reduced flow compared to a well without a mobile support. It follows that a mixture of open wells and loaded wells in a membrane reactor would have uneven flow. In a biochemical reaction, an uneven flow may cause some pores to receive reagents in a non-uniform fashion. Non-uniform delivery of reagents may lead, at least, to a delay in performing reactions because a longer flow is necessary to deliver reagents to all the wells. More significantly, non-uniform delivery may cause errors in interpreting results. For example, some wells may receive more reagents than others and the excess or lack of reagents may change the results of a biochemical reaction. Spatially uneven flow through the array layer may also result in the lateral diffusion of reaction products from a reactive well (i.e., one comprising a mobile support) to a neighboring empty well, which can lead to cross-contamination, or bleeding.

The problem with uneven (non-uniform) flow can be significantly reduced by the use of a porous high flow resistance membrane element. The porous membrane layer can be positioned below the planar array layer to provide significant flow restriction in the membrane reactor. This flow restriction is useful in achieving uniform or near-uniform flow of reagents through the membrane reactor. In a preferred embodiment, the membrane is substantially permeable to aqueous solutions but is substantially impermeable to the mobile supports. This is possible, for example, if the pores are smaller than the mobile supports so that mobile supports cannot flow through.

The porous membrane may be configured in any number of ways to provide satisfactory flow resistance in conjunction with the planar array layer. The porous membrane may comprise pores that are less than one tenth ( 1/10) or less than one hundredth ( 1/100) the size of the wells in the planar array layer. The porous high flow resistance membrane, because of its small pores, will have a flow restriction that is about 10-fold or more, preferable about 100-fold or more, than that of the planar array layer. Because the porous high flow resistance membrane provides most of the flow restriction in a membrane reactor, the wells of the planar array layer, regardless of whether it comprises a mobile support, would provide only a small portion of the flow restriction.

In one embodiment, we contemplate that the porous high flow resistance membrane has an average pore size of between about 0.01 μm and 10 μm, preferably between about 0.01 μm and 5 μm, more preferably between about 0.01 μm and 0.5 μm and even more preferably between about 0.1 μm and 1 μm, or less than 0.1 μm. This is particularly the case when a symmetric membrane is used. In one embodiment, the pore size is about 0.2 μm and in another embodiment, the pore size is about 0.02 μm. Preferably, the high flow resistance membrane has a pore diameter that is less than 10%, or less than 1%, of the well diameter of the planar array. If the membrane porosity is asymmetric (i.e., an anisotropic membrane) then different pores sizes may also be used.

The porous membrane of the invention can comprise one or more suitable materials, including glass, quartz, ceramics, metal, and silicon as well as polymeric substrates, such as polyolefin, polyamide, polyimide, polyurea, polyether, polyether imides, polyether sulfone, polyurethane, polyethylene, polyester, polycarbonate, polyethyleneamine, polyethylene terephthalate, polyethylene naphthalate, polyglycol acrylate (PGA), polymethylmethacrylate, polyacrylonitrile, polyvinyl acetate, polyvinylchloride (PVC), polyvinylidene fluoride, vinyl polymer, polyvinylacetal resin, polydimethylsiloxane (PDMS), polysulfone, polypropylene, polybutadiene, phenol-formalin resin, cellulose acetate, regenerated cellulose, nitrocellulose, melamine resin, and copolymers thereof.

In another embodiment, membranes with altered surface chemistries may be used. For example, the porous membrane may restrict flow of aqueous materials by being composed of a hydrophobic material. In a preferred embodiment of a hydrophobic porous high flow resistance membrane, we contemplate using a 10 to 40 μm PTFE membrane. In another preferred embodiment, the membrane has a pore size of less than or about 20 μm. Additionally, membranes with altered surface chemistries may be used (e.g., hydrophobic membranes).

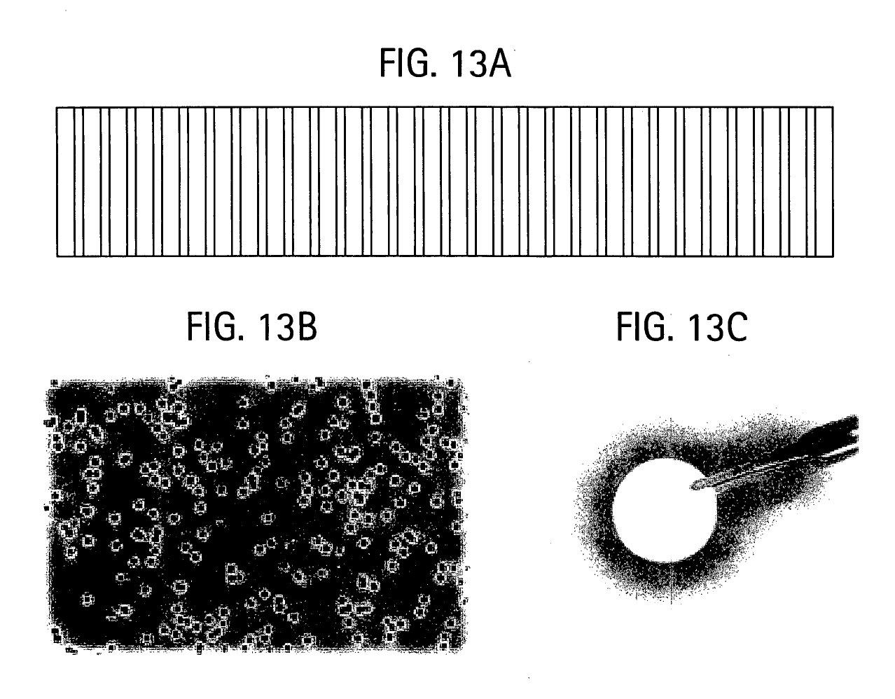

Preferably, the planar array is fabricated on a porous membrane. The surface of the membrane is preferably flat, with pores distributed in the membrane (e.g., FIGS. 13A-13C). Commercially available membranes can be used. Pore size and density can be chosen from different commercial products and used to adjust flow resistance. In preferred aspects, the pores are about 0.2 to 12 μm in a diameter. The pores can be oriented in a perpendicular direction to the membrane surface. Pores can be arranged uniformly, or in colonies or clusters at different areas on the membrane. Pores can also be distributed in a random manner. One or more additional porous membranes can be placed underneath the original porous membrane to increase flow resistance. Other structures can be placed underneath the porous membrane for additional support (see below).

Each layer of the membrane reactor can be offset or aligned to produce different three-dimensional structures. The structures for the planar array can be built into wedges or other tapered shape on the top surface of the membrane. Metal deposition can be used to coat the surface of the membrane. Structures for the planar array can be constructed on top and bottom sides of the porous membrane. The structures can be aligned in a single membrane or from membrane to membrane. In a later case, multiple membranes can be stacked together to form a more complex structure. Pores in the membrane can be partially blocked during metal deposition and electroplating. Pores can also be completely blocked in certainly areas with particular shapes. In certain aspects, the metal film can be applied to the membrane so as to avoid blocking the pores. For example, pores can be blocked by a masking process prior to metal deposition. The conductive, thin metal film on the membrane can provide a platform for the planar array layer as described in detail above.

Commercially available materials are contemplated for use with the invention. Preferred materials for the high-flow resistance membrane of the invention include nylon membrane filters such as those available from Millipore, including, but not limited to, Cat. # GNWP 025 00 and GNWP 047 00. Other preferred materials for the high-flow resistance include membrane ceramic filters such as those available from Refractron Technologies Corp. (Newark, N.Y.), including, but not limited to, alumina or silicon carbide filter plates with 15-30 μm pores and 40-50% porosity (volume %). Most preferred are track-etched membranes, such as Whatman Cyclopore™ polyester or polycarbonate membranes. For polyester membranes, pore sizes are about 0.1 to 5 μm and thickness is about 10 to 23 μm. For polycarbonate membranes, pore sizes are about 0.1 to 12 μm and thickness is about 10 to 20 μm. Most preferably, pore sizes are about 0.5 to 12 μm and thickness is about 9 to 23 μm. Metals for deposition include, but are not limited to, gold (Au), titanium (Ti), chromium (Cr), nickel (Ni), tin (Sn), copper (Cu), tantalum (TaN), aluminum (Al), palladium (Pd), platinum (Pt), zinc (Zn), silicon (Si), silver (Ag), and any alloy thereof, such as, Ag/Pd, Ag/Pt, Au/Sn, Ti/Pt/Au, TaN/Cu, and Al/Ti. Preferred metals for this aspect include nickel, chromium, and silver, as well as combinations comprising silver and chromium or silver and nickel. Gold as the top layer of coating is also preferred.

Optional Structural Support Layer

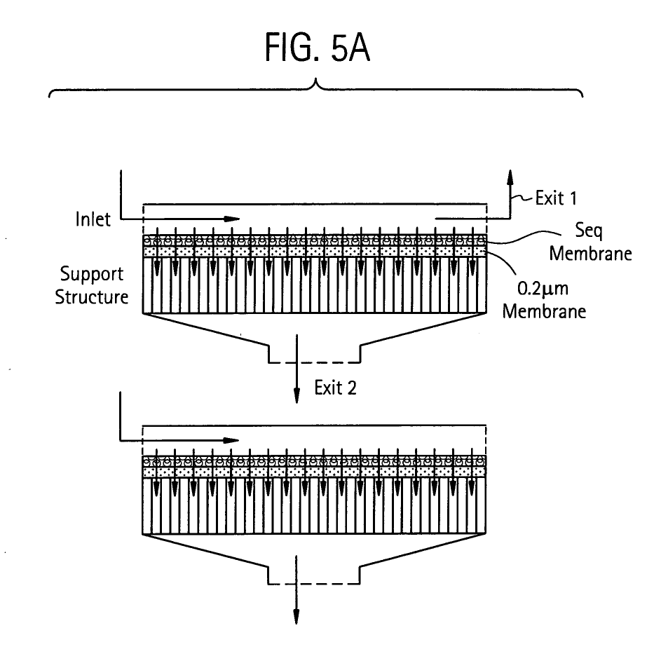

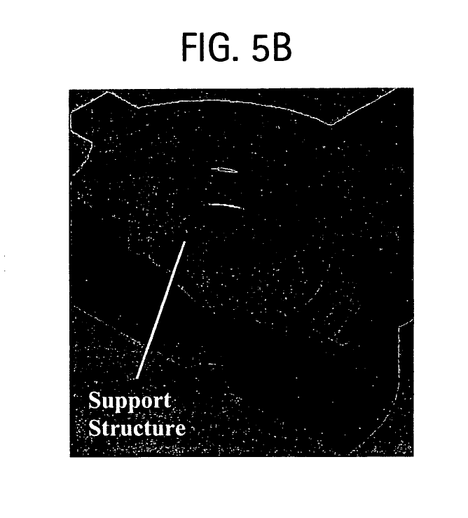

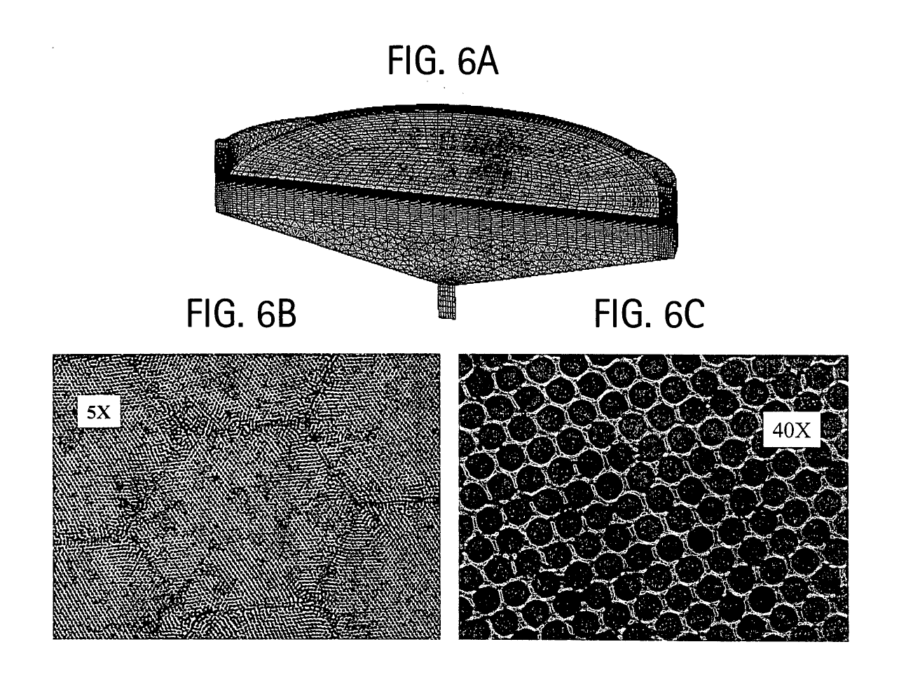

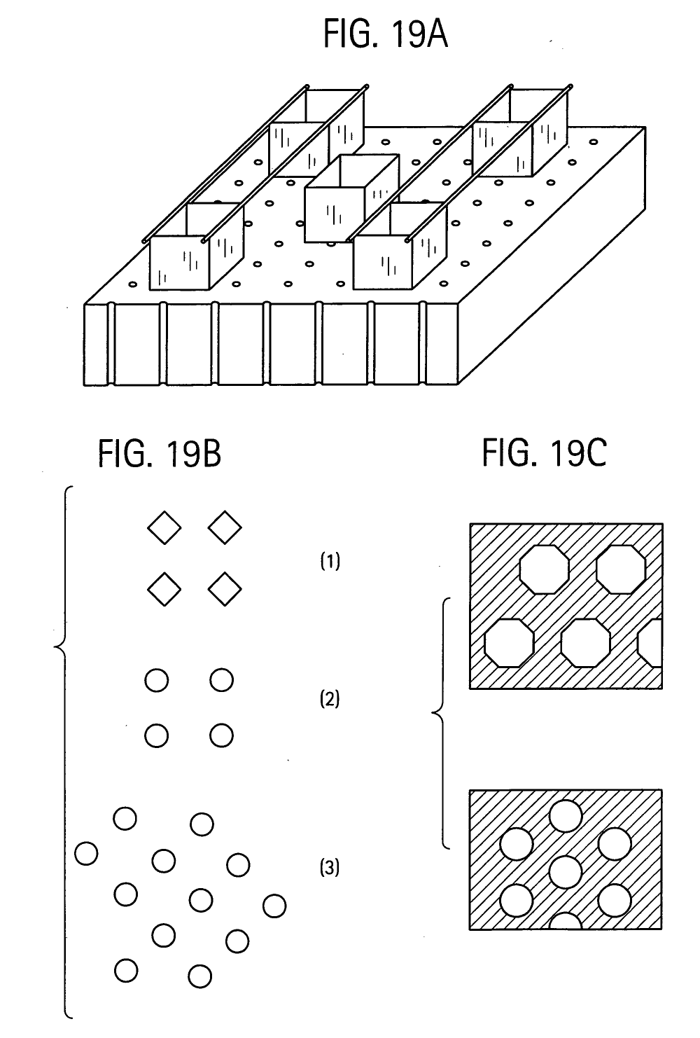

In addition to a planar array layer and a porous membrane, the membrane reactor can optionally employ a structural support layer that is more permeable than the other two layers and that is, in various embodiments, placed against and/or attached to the porous membrane or placed atop and/or attached to the planar array. This support layer can be used to provide mechanical support to membrane reactor. See, e.g., FIGS. 5 and 19 where examples of this are shown.

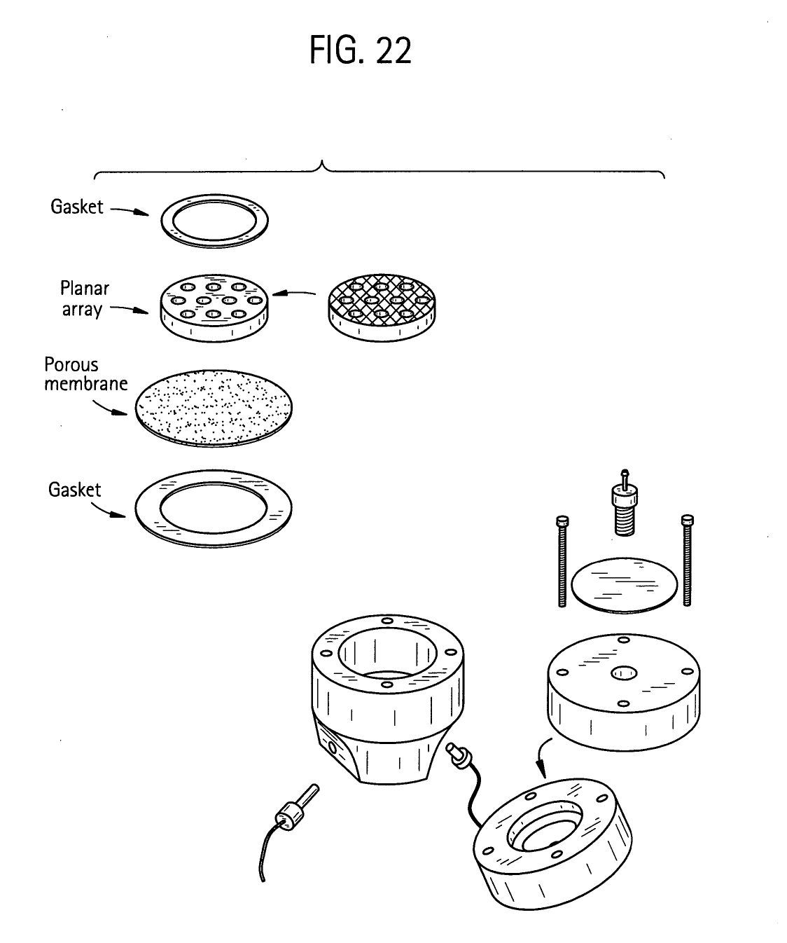

The support layer may be made from any material such as glass, metals, polymers, silicon, and/or ceramics with holes formed during manufacture (e.g., sintering, drilled by laser, cracking, etching, bombardment, and the like). It is noted that while a nonreactive material is generally preferred for the support layer, a nonreactive material is not necessary as long as the flow of reagents from the planar array layer to the porous support layer is sufficiently fast to prevent back diffusion of any molecules from the support layer to the planar array layer. In one preferred embodiment, we contemplate that the support layer comprises a metal mesh. In another embodiment, the support layer comprises plated metal beams to reinforce the top surface of the planar array layer. Multiple support layers can also be used, e.g., gaskets, rivets, and washers, to form a larger support structure (e.g., FIG. 22).

In various embodiments, commercially available materials can be used for the support layer. Preferred materials include stainless steel microfiltration meshes, such as Spectra/Mesh® from Spectrum Laboratories (Rancho Dominguez, Calif.), including, but not limited to, Cat. # 145827, 145936, 145826, and 145935.

Membrane Reactor Configuration

It should be appreciated that the membrane reactor can be constructed in a number of different configurations. For example, the porous high flow resistance membrane may be positioned on the top or the bottom of the planar array layer. In addition, two porous high flow resistance membranes may be utilized under the planar array layer or with the planar array layer between them. In addition, the permeable structural support layer may be positioned in several different configurations, e.g., on the top, bottom or in the middle of the membrane reactor. As will be appreciated, the structural support layer is optional and may not be required when the membrane reactor is configured with sufficient inherent support (e.g., when the membrane reactor is provided with additional support by being affixed to a circumferential support, much like a drum head).

In many cases it will be appropriate to consider the entire array assembly (i.e., the combination of porous membrane element plus planar array element) as a single substantially two-dimensional structure comprised of either an integral or a physical composite, as described further below. The membrane reactors of the present invention will be seen to possess some of the general structural features and functional attributes of commercially available microtiter filter plates of the sort commonly used in biology laboratories, wherein porous filter disks are molded or otherwise incorporated into the bottoms of plastic wells in 96-well plates. However, the membrane reactor is differentiated from these by the unparalleled high density of discrete reaction sites that it provides, by its unique construction, and by the novel and uniquely powerful way in which it can be operated to perform high throughput chemistries—for example, DNA amplification and/or DNA analysis.

The composite microreactor/filter structure (i.e., the membrane reactor of the present invention) can take several physical forms; as alluded to above. Two such forms are represented by physical composites and integral composites, respectively. The two functional elements of the structure include the planar array layer and the porous high flow resistance membrane. These elements may be provided as separate parts or components that are merely laid side-by-side, pressed together, or otherwise attached in the manner of a sandwich or laminate. This structural embodiment will be referred to hereinafter as a “physical composite”. Additional permeable supports (e.g., fine wire mesh or very coarse filters or metal beams) and/or spacing layers may also be provided where warranted to provide mechanical support. Plastic mesh, wire screening, molded or machined spacers, or similar structures may be provided atop the membrane reactor to help provide spatial separation between tangential flow of fluid across the top of the membrane reactor and the upper surface of the membrane reactor. Similar structures may be provided beneath the membrane reactor to provide a pathway for egress of fluid that has permeated across the membrane reactor.

In contrast to the operation of many previous microreactor arrays, wherein movement of reagents and reactants occurs solely by diffusion, the operation of the membrane reactors of the present invention employs a convective flow through the membrane reactor. In particular, a pressure difference is applied from the top to the bottom surface of the membrane reactor sufficient to establish a controlled convective flux of fluid through the structure in a direction normal to the substantially planar surface of the structure. Fluid is thus made to flow first through the planar array element and then subsequently across the porous membrane element. This convective flow enables the rapid delivery to the site of reaction of reagents and reactants and the efficient and complete removal of excess or unreacted components from the site of the reaction. Particularly important is the fact that the convective flow serves to impede or substantially prevent the back-diffusion of reaction products out of the upstream ends of the microchannels, where otherwise they would be capable of contaminating adjacent or even distant microreactor vessels.

In certain reaction systems of interest (e.g., DNA analysis by pyrophosphate sequencing, as discussed in more detail below), it may be necessary to avoid covalently immobilizing certain macromolecular reagents altogether. The DNA polymerase used in pyrophosphate sequencing is a case in point. It is believed that DNA polymerase should retain at least a certain degree of mobility if it is to function optimally. As a consequence, this particular enzyme must normally be treated as a consumable reagent in pyrophosphate sequencing, since it is not desirable to covalently immobilize it and reuse it in subsequent pyrophosphate sequencing steps. In the present invention, a polymerase may be in a native form or “tagged” with a moiety that adheres to the mobile supports, such as biotin. For the polymerase, we contemplate: a) placing polymerase in contact with template loaded mobile supports; b) flowing polymerase over the array; and c) both (i.e., employing (a) and (b) together). The present invention thereby provides means for localizing this macromolecular reagent within the microchannels or microvessels of a membrane reactor without having to covalently immobilize it.

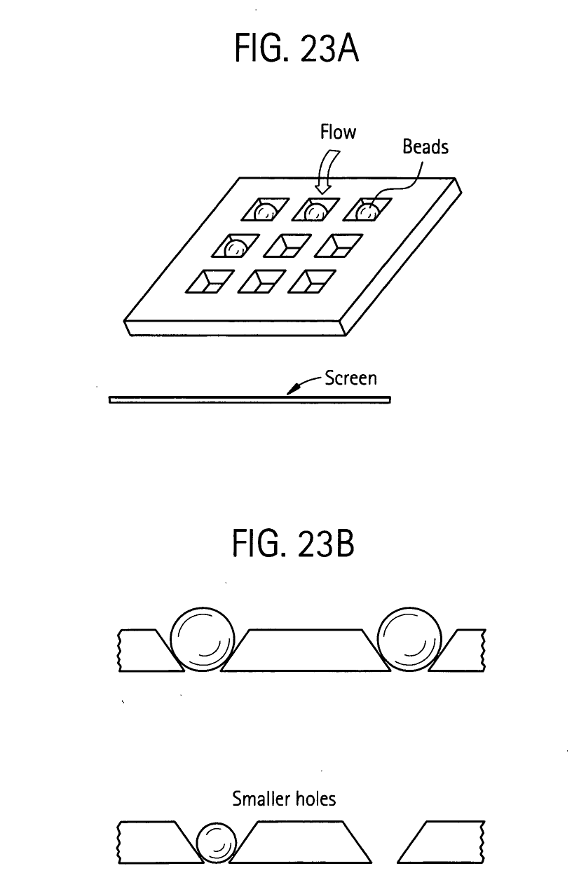

In preferred aspects, the membrane reactor is highly rigid and flat with precise control of well size, pitch, and reference anchors. The flat surface provides a good platform for optical focusing during the imagining process. The variation in well profile (e.g., size, shape, pitch) allows microfluidic control and specific flow distribution. Preferably, the flow resistance of the membrane reactor is less than 10 psi. Adjustable pitch and/or tapered wells also allow different loading densities for mobile supports (e.g., FIGS. 23A-23B). Precisely controlled sidewalls (e.g., height and profile) eliminate chemical crosstalk and improve trapping of mobile supports. Smooth sidewalls and bottoms concentrate and reflect light from the wells. Opaque sidewalls and bottoms eliminate or reduce optical crosstalk. The membrane reactor encompasses automatic or semi-automatic deposition of mobile supports. Plated metal beams are included to reinforce membrane strength and stability.

The membrane reactor is preferably made from commercially available materials using conventional micromachining methods. In preferred aspects, the membrane reactor is produced by batch processing with small bench top instruments, and the reactor is reused or disposed after each use. In one aspect of the invention, microfabrication facilities with clean rooms are used for at least part of the construction of the membrane reactor. The photomasks can be designed and drawn using available software. Metal deposition, photolithography, and electroplating can be performed by commercial vendors.

Applications of the Membrane Reactor

Many different types of reactions can be performed in a membrane reactor. In one embodiment, each cavity or well of the array comprises reagents for analyzing a nucleic acid or protein. Not all wells are required to include a nucleic acid or protein target. Typically those wells that comprise a nucleic acid comprise only a single species of nucleic acid (i.e., a single sequence that is of interest). There may be a single copy of this species of nucleic acid in any particular well, or they may be multiple copies.

It is generally preferred that a well comprise at least 1,000,000 copies of the species of nucleic acid sequence of interest, preferably between about 2,000,000 and 20,000,000 copies, and most preferably between about 5,000,000 and 15,000,000 copies of the species of nucleic acid sequence of interest. In one embodiment the nucleic acid species is amplified to provide the desired number of copies using polymerase chain reaction (“PCR”) (preferred), rolling circle amplification (“RCA”), ligase chain reaction, other isothermal amplification, or other conventional means of nucleic acid amplification. In one embodiment, the nucleic acid is single stranded. In other embodiments, the single stranded DNA is a concatamer with each copy covalently linked end to end.

The nucleic acid may be immobilized in the well, either by attachment to the well itself or preferably by attachment to a mobile support (e.g., a bead) that is delivered to the well. A bioactive agent (e.g., a sequencing enzyme) can be delivered to the array by dispersing it over the array to a plurality of mobile supports, wherein each mobile support has at least one reagent immobilized thereon, and wherein the reagent is suitable for use in a nucleic acid sequencing reaction.

The array can also include a population of mobile supports disposed in the wells, each mobile support having one or more bioactive agents (e.g., nucleic acids or sequencing enzymes) attached thereto. The diameter of each mobile support can vary. It is preferred that the diameter of the mobile support is such that only one mobile support is trapped within a single well in the planar array. Not every well in the planar array need comprise a mobile support. There are numerous contemplated embodiments; in one embodiment, at least 5% to 20% of the wells can have a mobile support; a second embodiment has about 20% to 60% of the reaction chambers can have a mobile support; and a third embodiment has about 50% to 100% of the reaction chambers with a mobile support. Preferably, the percentage of wells loaded with mobile supports is about 5%, 10%, or 25%.

By applying perpendicular fluidic flow, mobile supports carried in the stream can be pushed into wells in the planar array. Excessive mobile supports on top of the array can be flushed away with a parallel flow of fluid along the surface of membrane. Thus, the loading process for mobile supports can use perpendicular and parallel flows: the first flow pushes mobile supports into wells, while the latter flow pushes excessive mobile supports into empty well or off the array. In other aspects, mobile supports can be loaded using fluidic streams, vibration, shaking, rocking, spinning, centrifugation, or any combination thereof.

A mobile support typically has at least one reagent or reactant immobilized thereon. For the embodiments relating to pyrophosphate sequencing reactions or more generally to ATP detection, the reagent may be a polypeptide with sulfurylase or luciferase activity, or both. Alternatively, enzymes such as hypoxanthine phosphoribosyltransferase, xanthine oxidase, uricase or peroxidase could be utilized (e.g., Jansson and Jansson (2002), incorporated herein by reference). The mobile supports can be used in methods for dispersing over the array a plurality of mobile supports having one or more nucleic sequences or proteins or enzymes immobilized thereon.

In another aspect, the invention involves an apparatus for simultaneously monitoring the array of wells for light generation, indicating that a reaction is taking place at one or more particular sites. In this embodiment, the wells are sensors, adapted to comprise analytes and an enzymatic or fluorescent means for generating light in the wells. Such sensors are suitable for use in a biochemical or cell-based assays. The apparatus also includes an optically sensitive device to detect light from a well at a particular region of the optically sensitive device. The apparatus also includes means for determining the light levels detected at these particular regions and means for recording the variation of the light levels with time for each well.

In one specific embodiment, the instrument includes a light detection means having a light capture means and a fiber optic bundle for transmitting light to the light detecting means. We contemplate one light capture means to be a CCD camera. The fiber optic bundle is typically in optical contact with the array, such that light generated in an individual well is captured by a separate fiber or groups of separate fibers of the second fiber optic bundle for transmission to the light capture means.

The membrane reactor can be utilized to achieve highly parallel sequencing without electrophorectic separation of DNA fragments and associated sample preparation. The membrane reactor can also be used for other uses, e.g., combinatorial chemistry. For detection purposes, an array of photodetectors is utilized for monitoring light producing reactions within the membrane reactor. In a preferred embodiment, the array of photodetectors is a CCD camera. Another method of detection of discrete reactions within the membrane reactor is to monitor changes in light absorption as an indicator of a chemical reaction in a membrane reactor using an array of photodetectors.

Sequencing DNA by Pyrophosphate Detection

The methods and apparatuses described are generally useful for any application in which the identification of any particular nucleic acid sequence is desired. For example, the methods allow for identification of polymorphisms, including single nucleotide polymorphisms (SNPs) and haplotypes, and for transcript profiling. Other uses include sequencing artificial DNA constructs to confirm or elicit their primary sequence, and identifying specific mutant clones from random mutagenesis screens. The methods can also be used to determine cDNA sequences from single cells, whole tissues, or organisms from any developmental stage or environmental circumstance in order to determine a gene expression profile from that specimen. In addition, the methods allow for the sequencing of PCR products and/or cloned DNA fragments of any size isolated from any source. The DNA may be genomic DNA, cDNA, or recombinant DNA and may be derived from viral, bacterial, fungal, mammalian, or preferably human sources.

Sequencing of DNA by pyrophosphate detection (i.e., pyrophosphate sequencing) is described in various patents (Hyman, 1990, U.S. Pat. No. 4,971,903; Nyren et al., U.S. Pat. Nos. 6,210,891 and 6,258,568 and WO 98/13523; Hagerlid et al., 1999, WO 99/66313; Rothberg, U.S. Pat. No. 6,274,320 and WO 01/20039) and publications (Hyman, 1988; Nyrén et al., 1993; Ronaghi et al., 1998, Jensen, 2002; Schuller, 2002). The contents of the foregoing patents, patent applications and publications cited here are incorporated herein by reference in their entireties.

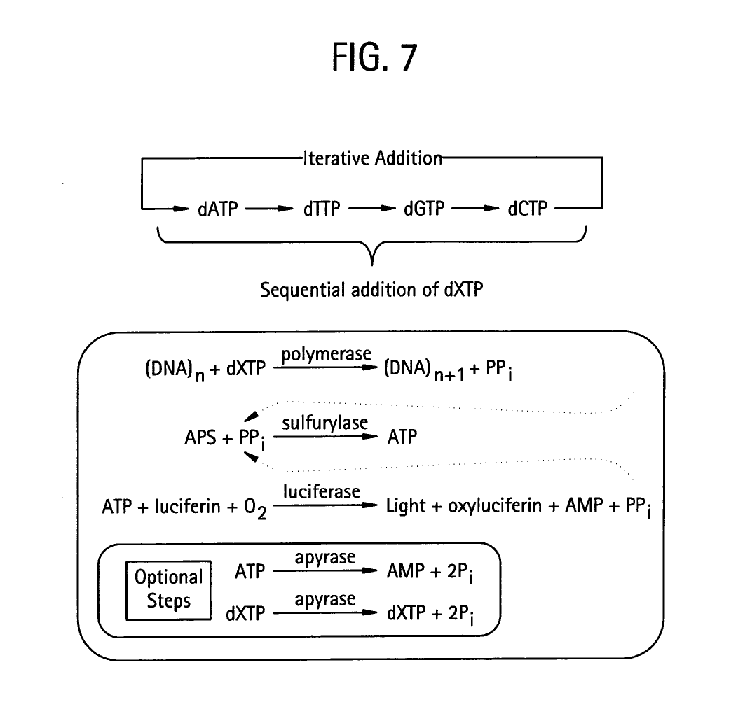

Pyrophosphate sequencing is a technique in which a complementary oligonucleotide is hybridized and extended using an unknown sequence (the sequence to be determined) as the template. This technique is also known as “sequencing by synthesis”. Each time a new nucleotide is polymerized onto the growing complementary strand, a pyrophosphate (PPᵢ) molecule is released. The release of pyrophosphate is then detected. The method involves iterative addition of the four nucleotides (dATP, dCTP, dGTP, dTTP) or of analogs thereof (e.g., α-thio-dATP). The time and extent of pyrophosphate release is monitored to permit identification of each nucleotide that is incorporated into the growing complementary strand. A schematic of pyrophosphate based sequencing is shown in FIG. 7.

Pyrophosphate can be detected via a coupled reaction in which pyrophosphate is used to generate ATP from adenosine 5′-phosphosulfate (APS) through the action of the enzyme ATP sulfurylase. The ATP is then detected photometrically via light released by the enzyme luciferase, for which ATP is a substrate. It may be noted that luciferase is capable of using dATP as a substrate. To prevent light emission on addition of dATP for sequencing, a dATP analog such as α-thio-dATP can be substituted for dATP as a sequencing nucleotide. The α-thio-dATP molecule can be incorporated into the growing DNA strand, but not used a substrate for luciferase.

Pyrophosphate sequencing can be performed in a membrane reactor in several different ways. One such protocol follows:
(1) A sample DNA is captured (preferably, many copies of a single sequence) onto beads and the beads are loaded onto the membrane reactor;
(2) As a negative control, a separate set of beads is prepared as in step (1) with the exception that no DNA is captured. The negative control is useful, at least, for determining background signal levels. All subsequent steps can be-performed in parallel using DNA loaded beads and negative control beads;
(3) (a) Luciferase and sulfurylase are loaded onto beads and dispose the loaded beads onto the membrane reactor. The loading step is performed using any suitable method for attaching proteins to bead surfaces which are known in the art; (b) DNA polymerase (e.g. Klenow fragment) is loaded onto the membrane reactor, which can be done concurrently with step (a) above; and
(4) A mixture of dXTP, APS (a substrate for sulfurylase), and luciferin (a substrate for luciferase) is flowed through the membrane reactor, cycling through the four nucleotides (dCTP, dGTP, dTTP, a-thio-dATP, or any suitable dATP analog) one at a time. It will be noted that these are all low-molecular weight molecules, so they will pass through the membrane reactor without undergoing appreciable concentration or polarization.

With the upstream-to-downstream flow of fluid into and through the membrane reactor:
(a) The appropriate dXTP (dATP, dTTP, dGTP or dCTP) is added by the polymerase and PPᵢ is produced in the region of the DNA being sequenced (with APS and luciferin flowing through);
(b) ATP is produced from APS and PPᵢ when PPᵢ is brought into contact with the sulfurylase enzyme (with luciferin flowing through); and
(c) Light is produced from ATP and luciferin in the vicinity of the luciferase enzyme.

Light production is monitored by a photodetector which is discussed in more detail as follows. As one example, a CCD camera can be optically coupled by a lens or other means to the membrane reactor. This can be used to monitor light production simultaneously from many wells or discrete reaction sites. CCD cameras are available with millions of pixels, or photodetectors, arranged in a 2-D array. Light originating from one well or discrete reaction site in or on a membrane reactor can be made to transmit a signal to one or a few pixels on the CCD. If each well or reaction site is arranged to comprise an independent sequencing reaction, each reaction can be monitored by one or at most a few CCD elements or photodetectors. By using a CCD camera or other imaging means comprising millions of pixels, the progress of millions of independent sequencing reactions can be monitored simultaneously.

In various aspects, a plurality of wells or reaction sites can comprise amplification products from a single sample of DNA. If different wells (or mobile support disposed therein) hold the amplification products of DNA samples, then the simultaneous sequencing of millions of different samples of DNA is possible. The distribution of DNA to be sequenced can be accomplished in many ways, two of which follow. In one approach, the amplification products of a single DNA strand are attached to a bead, and beads from many independent amplification reactions are combined and placed onto a membrane reactor. In another approach, many different strands of DNA are added in dilute concentration and applied to the membrane reactor. Each strand of DNA is attached to a different bead such that a plurality of wells or discrete reaction sites comprise only a single strand of DNA. The DNA is amplified within or upon the membrane reactor through a series of reactions. The DNA is then sequenced via addition of the reagents described above. One exemplary technique for amplification of DNA within the pores of a membrane reactor is described below (see Example 3).

Delivery of the DNA to be sequenced and the enzymes and substrates necessary for pyrophosphate-based sequencing can be accomplished in a number of ways. In a preferred embodiment, one or more reagents or reactants are delivered to the membrane reactor immobilized or attached to a population of mobile supports, e.g., beads, particles, or microspheres. The mobile support need not be spherical; in some aspects, hexagonal or irregular shaped beads may be used. The beads are typically constructed from numerous substances, e.g., plastic, glass, or ceramic, and cross-linked agarose gel. The mobile support of the invention may comprise various chemistries, such as, for example, methylstyrene, polystyrene, acrylic polymer, latex, paramagnetic, thoria sol, carbon graphite and titanium dioxide. The construction or chemistry of the mobile support can be chosen to facilitate the attachment of the desired reagent or reactant. In a preferred embodiment, the mobile supports are magnetic or paramagnetic.

Mobile support sizes depend on the well size and width of the well. In a preferred embodiment, the diameter of each mobile support is chosen so that the mobile support cannot pass through the pores in the membrane layer. It particular embodiments, the mobile supports may be smaller than the wells in the planar array. However, the porous high flow resistance membrane layer can stop the mobile support from flowing through the membrane reactor. In a preferred embodiment, the mobile supports are sized so that only one mobile support can fit within a single well and where the spatial separation between two adjacent reaction chambers has a linear dimension of between about 5 μm and 200 μm, preferably between about 10 μm and 150 μm, more preferably between about 25 μm and 100 μm, more preferably between about 50 μm and 75 μm, and most preferably between about 20 μm and 35 μm. In a specific embodiment, the mobile support diameter may be 31 μm and the well diameter may be 33 μm. Even though the 31 μm mobile support may flow through the 33 μm well, the porous high flow resistance membrane layer prevents the mobile support from flowing through.

In some embodiments, a reagent immobilized to the mobile support can be a polypeptide with sulfurylase activity, a polypeptide with luciferase activity, or both on the same or different mobile supports, or a chimeric polypeptide having both sulfurylase and luciferase activity. In one embodiment, it can be an ATP sulfurylase and luciferase fusion protein (see, e.g., U.S. patent application Ser. No. 10/122,706, filed Apr. 11, 2002, and U.S. patent application Ser. No 10/154,515, filed May 23, 2002; which are incorporated herein by reference in their entirety). Other sulfurylase and/or luciferase that may be used include those described in U.S. Pat. Nos. 5,583,024; 5,674,713, and 5,700,673, and WO 00/24878; all incorporated herein in their entirety. Ultra-Glow luciferase (available from Promega) is also suitable for use with this invention.

In a preferred embodiment, both luciferase and sulfurylase are immobilized on the same mobile support. Since the product of the sulfurylase reaction is consumed by luciferase, proximity between these two enzymes may be achieved by covalently linking the two enzymes in the form of a fusion protein. Alternatively, a fusion protein combining functional polymerase, sulfurylase and luciferase activity may be used. In other embodiments, a reactant immobilized to the mobile support can be a nucleic acid whose sequence is to be determined or analyzed. A DNA or RNA polymerase can be incubated with mobile supports that have nucleic acids attached thereto.

Generally, a membrane reactor device having normal cross-flow exhibits high levels of wash efficiency. In some cases, the chamber cannot be washed efficiently within a reasonably short period of time. This can have a significant impact on the accuracy of pyrophosphate sequencing. In such situations, apyrase may be applied to degrade the leftover nucleotides after each nucleotide delivery. The use of apyrase is typically at concentrations of 1 U/l to 100 U/l preferably 4 U/l to 40 U/l more preferably 8 U/l to 20 U/l, most preferably 8.5 U/l. In some cases, high fidelity but low processivity polymerase (e.g., Klenow) may be used, and polymerase may be present in the flow. Preferably, the flow rate of the membrane reactor device is about 0.15 ml/minute/cm² to 4 ml/minute/cm², or about 0.1 ml/minute/cm² to 5 ml/minute/cm².

Bead Attachment Chemistry

In some embodiment, the bioactive agents (e.g., nucleic acids) are synthesized, and then covalently attached to the mobile supports. As appreciated by those of skill in the art, this depends on the composition of the bioactive agents and the mobile supports. The functionalization of solid support surfaces, e.g., polymers, with chemically reactive groups such as thiols, amines, carboxyls, etc., is generally known in the art. Accordingly, “blank” mobile supports may be used that have surface chemistries that facilitate the attachment of the desired functionality. Additional examples of these surface chemistries for blank mobile supports include, but are not limited to, amino groups including aliphatic and aromatic amines, carboxylic acids, aldehydes, amides, chloromethyl groups, hydrazide, hydroxyl groups, sulfonates, and sulfates.

These functional groups can be used to add any number of different candidate agents to the mobile supports, using well known chemistries. For example, candidate agents comprising carbohydrates may be attached to an amino-functionalized support. The aldehyde of the carbohydrate can be made using standard techniques. The aldehyde can then be reacted with an amino group on the surface of the mobile support. In an alternative embodiment, a sulfhydryl linker may be used. There are a number of sulfhydryl reactive linkers known in the art such as SPDP, maleimides, α-haloacetyls, and pyridyl disulfides (see for example the 1994 Pierce Chemical Company catalog, technical section on cross-linkers, pages 155-200, incorporated here by reference). These groups can be used to attach proteinaceous agents comprising cysteine to the support. Alternatively, an amino group on the candidate agent may be used for attachment to a suitable electrophilic moiety on the surface. Such moieties include, but are not limited to, NHS esters. As examples, a large number of stable bifunctional groups are well known in the art, including homobifunctional and heterobifunctional linkers (see Pierce Catalog and Handbook, pages 155-200).

In an additional embodiment, carboxyl groups (either from the surface or from the candidate agent) may be derivatized using well known linkers (see Pierce catalog). For example, carbodiimides may be used to activate carboxyl groups for attack by nucleophiles such as amines (see Torchilin et al., Critical Rev. Therapeutic Drug Carrier Systems, 7(4):275-308 (1991)). Proteinaceous candidate agents may also be attached using other techniques known in the art, for example for the attachment of antibodies to polymers; see Slinkin et al., Bioconj. Chem. 2:342-348 (1991); Torchilin et al., supra; Trubetskoy et al., Bioconj. Chem. 3:323-327 (1992); King et al., Cancer Res. 54:6176-6185 (1994); and Wilbur et al., Bioconjugate Chem. 5:220-235 (1994). It should be understood that the candidate agents may be attached in a variety of ways, including those listed above. Preferably, the manner of attachment does not significantly alter the functionality of the candidate agent. That is, the candidate agent should be attached in such a flexible manner as to allow its interaction with a target.

As one example, NH₂ surface chemistry beads can be used for immobilizing enzymes on beads. Surface activation is achieved with a 2.5% glutaraldehyde in phosphate buffered saline (10 mM) providing a pH of 6.9 (138 mM NaCl, 2.7 mM KCl). This mixture is stirred on a stir bed for approximately 2 hours at room temperature. The beads are then rinsed with ultrapure water plus 0.01% Tween 20 (surfactant), 0.02%, and rinsed again with a pH 7.7 PBS plus 0.01% Tween 20. Finally, the enzyme is added to the solution, preferably after being prefiltered using a 0.45 μm Amicon™ micropure filter. In a particularly preferred embodiment, the mobile supports and bioactive agents are linked using a biotin/streptavidin linkages, which are well known to those skilled in the art.

Apparatus for Detecting a Reaction in the Membrane Reactor:

The invention provides an apparatus for simultaneously monitoring an array of wells for light signals which indicate that one or more reactions are taking place at a particular well. The reaction event, e.g., photons generated by luciferase, may be detected and quantified using a variety of detection apparatuses, e.g., a photomultiplier tube, a CCD, CMOS, absorbance photometer, luminometer, charge injection device (CID), or other solid state detector, as well as the apparatuses described herein. In a preferred embodiment, the quantitation of the emitted photons is accomplished by the use of a CCD camera fitted with a fused fiber optic bundle. In another preferred embodiment, the quantitation of the emitted photons is accomplished by the use of a CCD camera fitted with a microchannel plate intensifier. A back-thinned CCD can be used to increase sensitivity. CCD detectors are described in, e.g., Bronks, et al., 1995. Anal. Chem. 65: 2750-2757. The CCD sensitivity may be enhanced by the known method of chilling the CCD during exposure.

An exemplary CCD system is a Spectral Instruments, Inc. (Tucson, Ariz.) Series 600 4-port camera with a Lockheed-Martin LM485 CCD chip and a 1-1 fiber optic connector (bundle) with 6-8 μm individual fiber diameters. This system has 4096×4096, or greater than 16 million pixels, and has a quantum efficiency ranging from 10% to >40%. Thus, depending on wavelength, as much as 40% of the photons imaged onto the CCD sensor are converted to detectable electrons.

The invention also provides a microimaging system for imaging one or more light emissions (e.g., from a pyrophosphate sequencing reaction) from a membrane reactor. Preferably, the system comprises two lens groups. The first lens group is the front lens group which is positioned closer to the light source to be detected to collect the light emitted. The second lens group is the rear lens group that is positioned closer to the light detector such as a CCD detection device to image the light onto the detector. In one aspect of the invention, the front lens group and rear lens group are identical.

In a preferred embodiment, the lens group comprises 50 mm lens with an aperture larger than 2.8 (e.g., 2.0, 1.8, 1.4, 1.0, etc.). Preferably, the lens group has a focal length of at least 30 mm, at least 50 mm, or at least 70 mm. In specific aspects, the lens group has an aperture brighter than or equal to 4.0, or brighter than or equal to 2.8. In other aspects, the lens group has a numerical aperture larger than 0.1, 0.2, or 0.3. It should be noted that the larger apertures are expressed by a smaller aperture value so that, for example, an aperture of 1 is larger than an aperture of 2. An exemplary imaging system is shown in FIG. 2.

The data from the optical detection device can be analyzed instantaneously or stored electronically (e.g., by computers, hard drives, optical drives, solid state memories) for subsequent analysis by methods known to those of skill in the art.

In an alternate embodiment, a fluorescent moiety can be used as a label and the detection of a reaction event can be carried out using a confocal scanning microscope. The microscope can be used to scan the surface of an array with a laser or other techniques such as scanning near-field optical microscopy (SNOM) which are capable of smaller optical resolution, thereby allowing the use of “more dense” arrays. For example, using SNOM, individual polynucleotides may be distinguished when separated by a distance of less than 100 nm, e.g., 10 nm×10 nm. Additionally, scanning tunneling microscopy (Binning et al., Helvetica Physica Acta, 55:726-735, 1982) and atomic force microscopy (Hanswa et al., Annu Rev Biophys Biomol Struct, 23:115-139, 1994) can be used.

Additional material may be found in U.S. application Ser. No. 10/191,438 filed Jul. 8, 2002, U.S. application Ser. No. 60/476,592 filed Jun. 6, 2003, U.S. application Ser. No. 60/476,602, filed Jun. 6, 2003, U.S. application Ser. No. 60/476,313 filed Jun. 6, 2003, U.S. application Ser. No. 60/476,504 filed Jun. 6, 2003, and U.S. Pat. No. 6,274,320. All patent applications and patents, listed in this disclosure, are hereby incorporated by reference in their entirety.

Many variations and alternative embodiments of the present invention as applied to DNA sequencing and other applications will be readily apparent and are considered to be within the scope of the present invention.

### EXAMPLES

The examples are presented in order to more fully illustrate the preferred embodiments of the invention. These examples should in no way be construed as limiting the scope of the invention, as illustrated in the appended claims.

### Example 1

### Preparation of Beads

Esterification of carboxyl derivative of sepharose beads is achieved with N-hydroxysuccinimide (NHS) and this leads to the formation of activated esters that react rapidly with primer containing amino-groups to give stable amide bonds. Beads to be used for this purpose are supplied (Amersham) in 100% isopropanol to preserve the activity prior to coupling. Twenty-five microliters of 1 mM amine-labeled HEG primer are dissolved in coupling buffer (200 mM NaHCO₃, 0.5 M NaCl, pH 8.3). Beads were activated by adding 1 ml of ice cold 1 mM HCl. Beads were washed two times with ice cold coupling buffer. Amine labeled primers and amine labeled biotin, in a ratio of 1:9 respectively) are added to the beads and incubated for 15 to 30 minutes at room temperature with rotation (to allow coupling to happen). Amine-labeled biotin is added. After coupling the emulsion PCR, the streptavidin is added to be coupled to the biotin. Then the biotinylated sulfurylase and luciferase (454 Life Sciences) are coupled to the streptavidin.

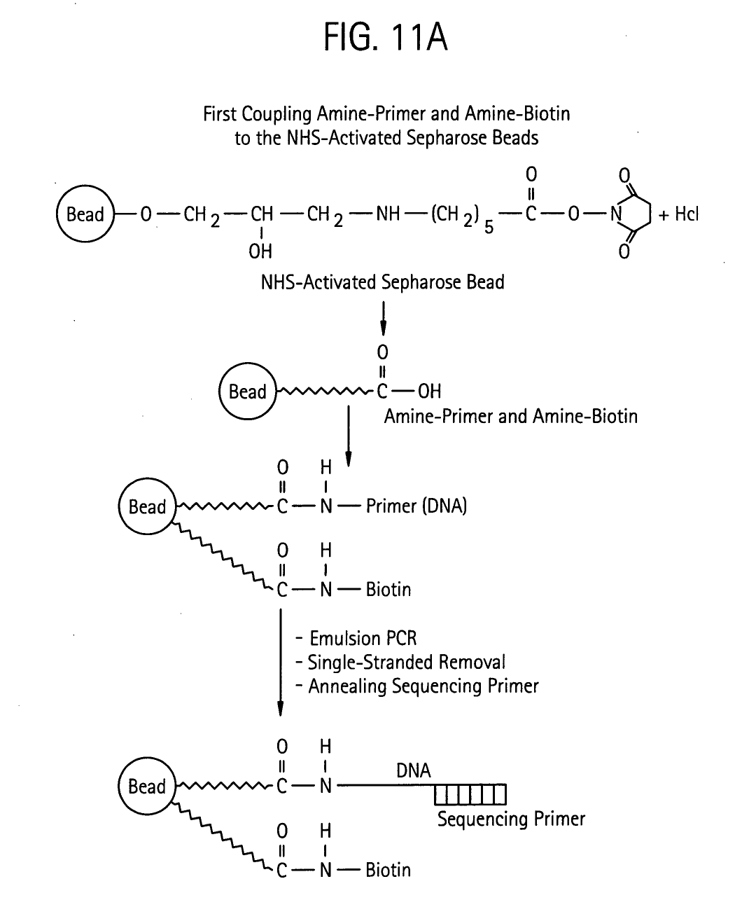

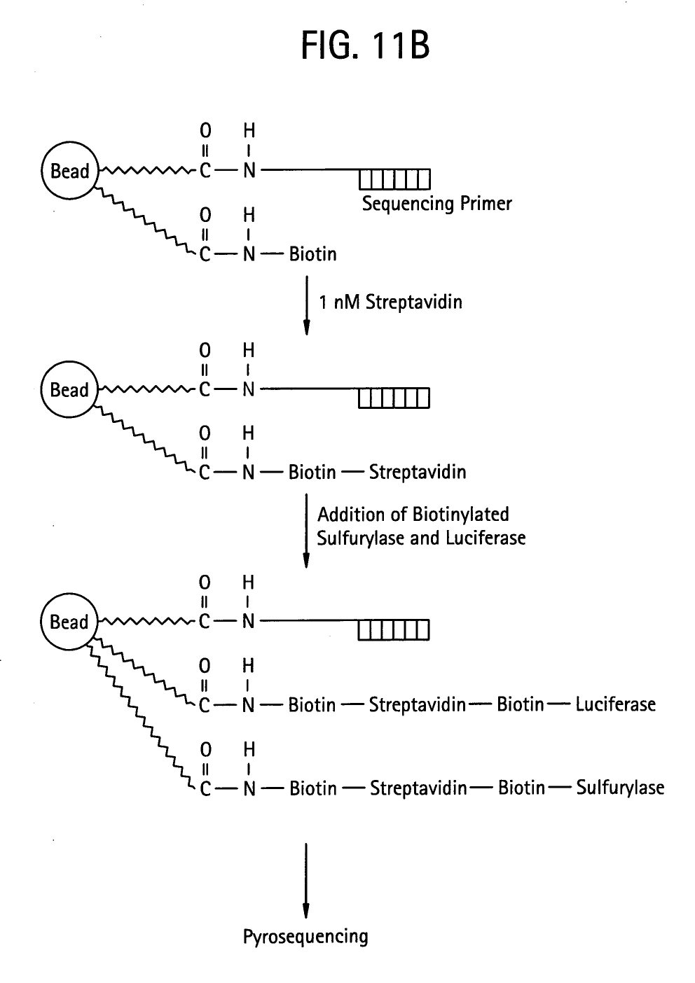

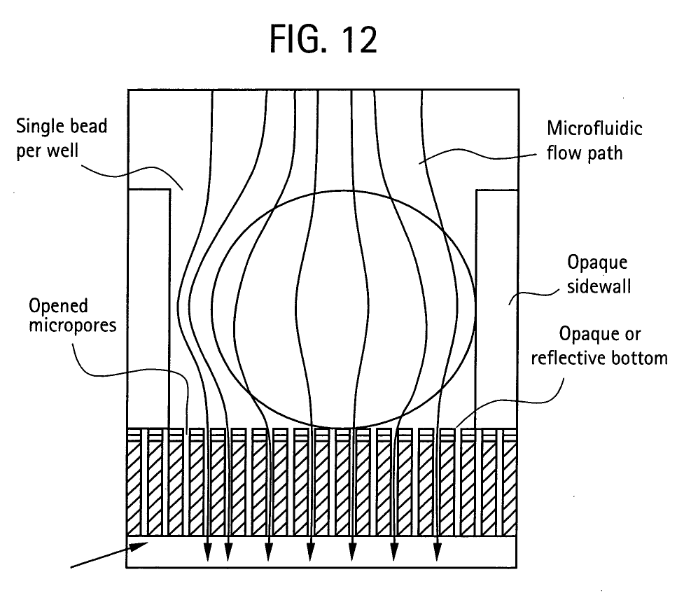

Then the beads were washed one time with coupling buffer. The beads were washed two times with Acetate buffer (0.1 M sodium acetate, 0.5 M NaCl, pH 4). The beads were washed three times with coupling buffer (0.5 M ethanolamine, 0.5 M NaCl, pH 8.3). The beads were incubated with 500 μl of blocking buffer for one hour with rotation at room temperature to allow for deactivation or blocking of any leftover active groups. The beads were washed with (a) coupling buffer and then with (b) acetate buffer. This wash ((a) then (b)) was repeated three times. The beads were washed two times in 1× annealing buffer. The annealing buffer also serves as the storage buffer. This procedure is illustrated in FIG. 11.

### Example 2

### Sequencing UATF9 DNA Template on Convective Rig

Loading the Beads

Streptavidin-sepharose beads were size-selected by filtering to obtain diameter between 30-36 μm. The primers and target DNA included: MMP7A sequencing primer (5′-ccatctgttc cctccctgtc-3′; SEQ ID NO:6); target DNA, termed UATF9 (3′-atgccgcaaa aacgcaaaac gcaaacgcaa cgcatacctc tccgcgtagg cgctcgttgg tccagcagag gcggccgccc ttgcgcgagc agaatggcgg tagggggtct agctgcgtct cgtccgggg-5′; SEQ ID NO:7); biotinylated primer and PCR reverse primer, termed Bio-Heg-MMP1(5′-5Bio//iSp18//iSp18//iSp18/cca tct gtt gcg tgc gtg ct-3′; SEQ ID NO:8); and PCR forward primer, termed MMP1A (5′-cgtttcccct gtgtgccttg-3′; SEQ ID NO:9). For the PCR reverse primer, “5Bio” indicates biotin and “iSp18” indicates Spacer 18.

The biotinylated PCR products were immobilized onto Streptavidin-Sepharose beads. Immobilized PCR product was incubated in 0.10 M NaOH for 10 min, and the supernatant was removed to obtain single-stranded DNA. The beads containing single-stranded DNA were washed 3 times with 100 μl of 1× Annealing Buffer, pH 7.5 (30 mM Tris-HCl, 3 mM magnesium acetate, from Fisher). The beads were pelleted by centrifugation for 1 min at a maximum speed of 13,000 rpm. Supernatant was removed and the beads were suspended in 25 μl of 1× Annealing Buffer. Five microliter of 100 pmol sequencing primer was added to mixture. The beads were incubated at 65° C. for 5 min and cooled to room temperature. The beads were washed 3 times with 100 μl of 1× Annealing Buffer and resuspended in final volume of 100 μl.

Loading the Beads into the Membrane and Assembly into the Loading Jig

The beads were resuspended at a concentration of about 3,500 beads per microliter in 1× Annealing Buffer. Following this, 25 μl of the suspension was added to 200 μl of 1× Assay Buffer, pH 7.8 (25 mM tricine (Fisher), 5 mM magnesium acetate (Fisher), 1 mM dithiothreitol, 0.4 mg/ml polyvinylpyrrolidone, 0.01% Tween 20, 1 mg/ml BSA, all from Sigma (St. Louis, Mo.)). In the loading jig, a 0.2 μm nylon membrane was placed below the 30 μm pore nylon membrane (Sefar). The loading jig was then attached to a peristaltic pump having a flow rate of 1 ml per min. Next, 200 μl of 1× Assay Buffer was used to wash the membrane. After washing, and while the membrane was partially dry, 200 μl of the bead suspension was added to the membrane. Negative pressure was applied to the membrane along with 500 μl of 1× Assay Buffer to force the beads onto the pores of the membrane.

After the application of beads, the membrane was washed with 500 μl of 1× Assay Buffer. The membrane was disassembled and placed in 1× Assay Buffer (e.g., 20 ml) in a test tube (e.g., Falcon) for storage. The following solutions were mixed in a container: 500 μl of biotinylated ATP Sulfurylase enzyme at 1 mg/ml (454 Life Sciences); 500 μl of biotinylated Luciferase at 3 mg/ml (454 Life Sciences); and 500 μl of 1× Assay Buffer. The bead-loaded nylon membrane was placed in the enzyme mixture. The mixture was rotated with the nylon membrane for 20 min at room temperature at a speed of one rotation per two seconds. The membrane was then placed in 20 ml of 1× Assay Buffer and swirled for 2 minutes. This wash step was repeated once. A solution of 970 μl 1× Assay Buffer with 30 μl of Bst Polymerase enzyme (New England Bio Labs) was prepared. The membrane was immersed in this solution, and the solution/membrane was rotated for 25 min at room temperature at a speed of one rotation per two seconds. After the rotations, the membrane was washed two times in 20 ml of 1× Assay buffer.

Preparing the Membrane for Sequencing on Convective Rig:

A 0.2 μm nylon membrane was immersed in 20 ml of 1× Assay Buffer and placed above the wire mesh on the sequencing Jig holder. A 30 μm membrane containing the DNA beads was placed on top of the 0.2 μm nylon membrane. A sequence Jig cover with an optical glass window (13 mm) was placed on top of the membranes. The Jig cover was tightly attached to the lower part of the membrane holder. The cover and holder were threaded and tightening was performed by screwing the two parts together. The sequencing Jig was placed on the z-translation stage below the CCD camera. The acquisition time on the camera was set to 7 sec and the read out time was set to 0.25 sec. The inlet of the membrane holder was connected to the outlet of the pump, which was connected to the Valco valve (Valco Instruments, Houston, Tex.). The lower part of the membrane holder was connected to the second peristaltic pump. The outlet of the sequencing chamber was connected to the waste.

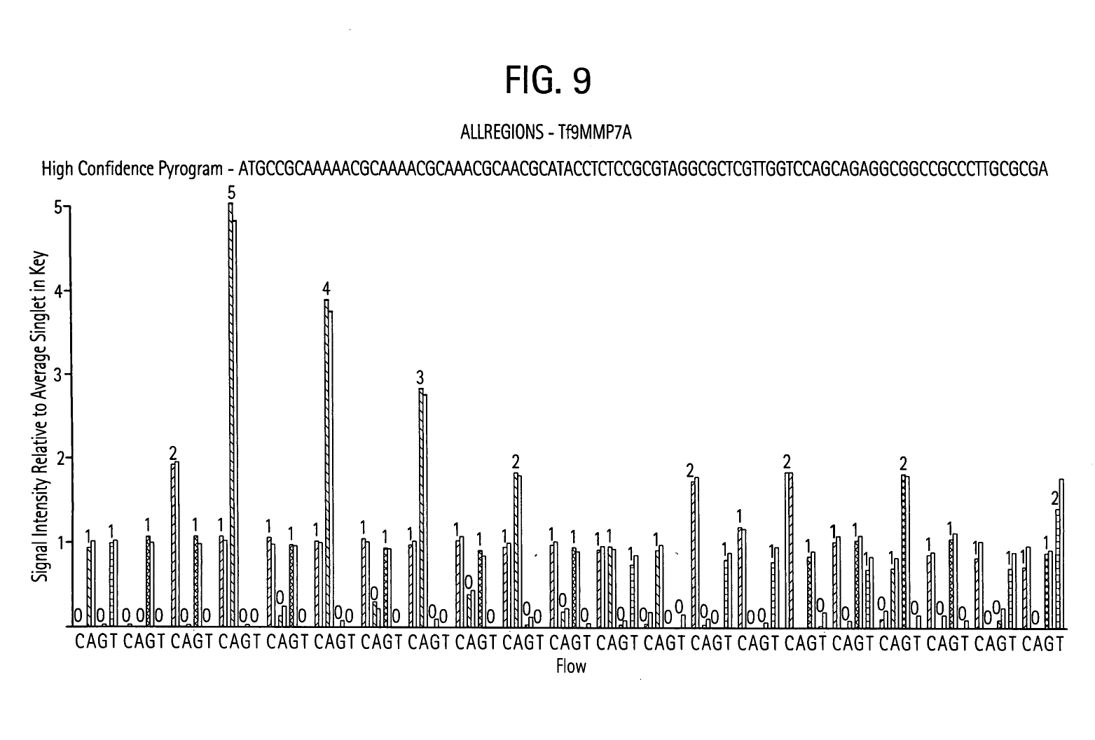

The substrate (25 mM tricine, 5 mM magnesium acetate, both from Fisher; 1 mM dithiothreitol, 0.4 mg/ml polyvinylpyrrolidone, 0.01% Tween 20, all from Sigma; 300 μM D-Luciferin, from Regis; 2.5 μM adenosine-5′-O-phosphosulfate, from Axxora, Inc.) was flowed for 2 min to prime the flow chamber and expel any air bubbles. This allowed the DNA on the nylon membrane to be equilibrated with substrate. The reagents were flowed through the chamber in the following order: 1) dCTP; 2) substrate; 3) nucleotide Sp-dATP-α-thio; 4) substrate; 5) dGTP; 6) substrate; 7) dTTP; and 8) substrate. This cycle was repeated 20 times. The flow rates for the lateral and vertical flow were controlled as follows. The nucleotide flow (for steps (1), (3), (5), and (7), above) was at 2 ml/min lateral for 21 sec and then 0.5 ml/min for 7 sec. The vertical flow was 0.5 ml/min for the same time. Total time was 28 sec. The substrate flow (for steps (2), (4), (6), and (8), above) was at 2 ml/min lateral flow and 1 ml/min vertical flow 1 ml/min for 77 sec. The results of the sequencing reaction are shown in FIG. 9.

### Example 3

### PCR on Nylon Membrane Containing Beads and Sequencing Using a Pyrophosphate Sequencer

The sequencing step was used to confirm the fidelity of the amplified template. The primers and probe included:

| | | SEQ |
|---|---|---|
| | | ID |
| PRIMER | SEQUENCE | NO: |
| Adeno P1 | 5′ caa tta acc ctc act aaa gg 3′ | 1 |
| forward | | |
| Adeno P2 | 5′ gta ata cga ctc act ata ggg 3′ | 2 |
| reverse | | |
| tf2 | 3′cgatcaagcgtacgcacgtggttgttaaagc | 3 |
| | ttttttgaaagttaatctcctggttcaccgtctg | |
| | ctcgtatgcggttaccaggtcggcggccgccacg | |
| | tgtgcgcgcgcgggactaatcccggttcgcgcgt | |
| | cgg 5′ | |
| Biotinylated | 5′/Bio//iSp18//iSp18/iSp18/caa tta | 4 |
| probe | acc ctc act aaa gg 3′ | |
| Adeno P1 | | |

The sepharose beads were treated as in Example 2, with a concentration of 3,500 beads per microliter. Next, 90 μl of sepharose beads were washed by resuspension in 200 μl of 1× PCR buffer and this was followed by centrifugation for a total of three washes. After the final wash, 200 μl of 1× PCR buffer was placed on top of the beads pelleted by centrifugation. Then, 6 μl of 100 pmol/μl biotinylated P1 probe was added to the top of the beads/PCR buffer. The beads were resuspended and the tube containing the beads was placed on a rotator for 45 min at room temperature. Following the rotation, the beads were washed three times with 200 μl 1× PCR buffer. A 15 μ; aliquot of bead suspension was placed in a microcentrifuge tube and briefly centrifuged for 30 sec at 13.2k rpm to let the beads settle down. This sample was marked “Sample A”. Another sample, “Sample B” was prepared the same way.

The aqueous layer above the beads in Sample A (negative control) was removed and replaced with 50 μl of PCR mix (37.7 μl H₂O, 5 μl 10× PCR buffer, 1 μl dNTPs (10 mM each), 0.4 μl of 100 pmol/μl P1 forward primer, 0.4 μl of 100 pmol/μl P2 reverse primer, 5 μl Betaine (5 M), and 0.5 μl of Taq polymerase (5 U/μl). Nylon membranes were cut into 2 mm circles using a die cutter. The circles were pre-wetted by immersion in 1× PCR buffer. One 2 mm circle of nylon membrane was immersed in Sample A such that the beads were attached to the membrane; filling most of the pores. Sample A and the nylon membrane were placed on a rotator for 3 hours at 4° C. to allow the beads to take up the components of the PCR mixture. The nylon membrane was removed and fully immersed in a tube containing about 20 μl of mineral oil.

The aqueous layer above the beads in Sample B was replaced with 50 μl PCR mix (35.45 μl H₂O, 5 μl 10× PCR buffer, 1 μl dNTPs (10 mM each), 0.4 μl of 100 pmol/μl P1 forward primer, 0.4 μl of 100 pmol/μl P2 reverse primer, 5 μl Betaine (5 M), 0.5 μl Taq polymerase (5 U/μl) and 2.25 μl of 1.67 attomol/μl tf2 adeno fragment. One attomole is defined as 1×10⁻¹⁸ moles. The estimated concentration of DNA per bead prior to amplification was 10 copies per bead. (Note that where each bead was to contain a distinct template sequence, then a maximum of one unique sequence per bead was preferred). One 2 mm circle of nylon membrane was immersed in Sample B such that the beads were attached to the membrane; filling most of the pores. Sample B and the nylon membrane were placed on a rotator for 3 hours at 4° C. to allow the beads to take up the components of the PCR mixture. The nylon membrane was removed and fully immersed in a tube containing about 20 μl of mineral oil.

The tube containing the nylon membrane with Sample A and the tube containing the nylon membrane with Sample B were placed in a thermocycler with the following reaction conditions. Step 1: incubation at 96° C. for 2 min; Step 2: incubation at 96° C. for 1 min; Step 3: incubation at 58° C. for 1 min; Step 4: incubation at 72° C. for 1 min, go to Step 2, 29 times; Step 5: incubation at 72° C. for 10 min; Step 6: incubation at 14° C. overnight or until the reaction was terminated. After the PCR reaction, the PCR tubes with the nylon membranes were removed from the thermocycler. The membranes were removed and placed into separate tubes containing 1 ml chloroform and 200 μl of 1× Annealing Buffer. The tubes were shaken several times. The membranes were transferred to individual tubes containing 1 ml of chloroform and the tubes were rotated several times. The membranes were then transferred to individual tubes with 200 μl of 1× Annealing Buffer. The tubes were then rotated several times. This procedure was repeated an additional two times with 200 μl of Annealing Buffer.

To denature DNA on the beads, the membranes were transferred to individual tubes with 50 μl of 1× Annealing Buffer. The tubes were heated to 90° C. for 2 min in a PCR thermocycler. Next, the membranes were transferred to individual tubes containing 50 μl of 1× Annealing Buffer on ice. The membranes were washed two times with 100 μl of 1× Annealing Buffer. The membranes were then incubated with a solution of 5 μl of 100 pmol of P2 primer mixed with 20 μl of 1× Annealing Buffer. The tubes were placed in a thermocycler and heated to 65° C. Next, the tubes were slowly cooled to room temperature to allow the P2 sequencing primer to anneal to the DNA template. Following annealing, the membranes were washed two times with 100 μl of 1× Annealing Buffer.

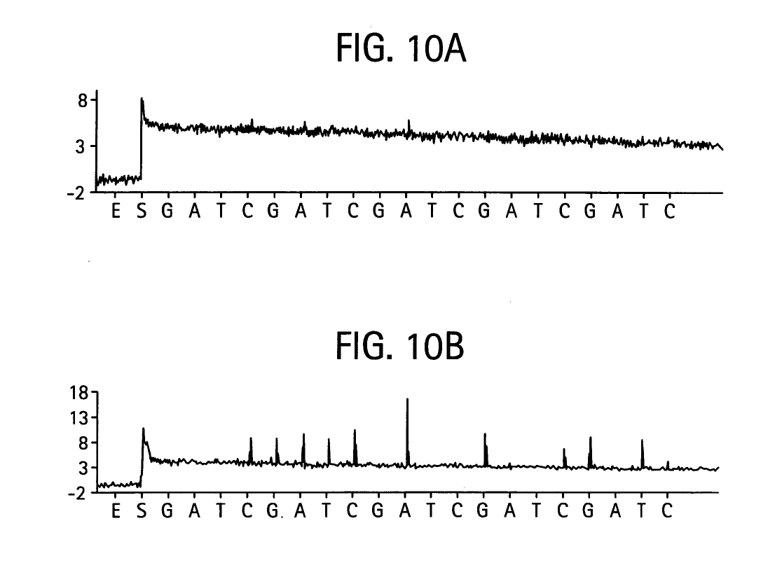

To confirm the fidelity of the amplified DNA fragment, the reaction product was sequenced on the beads using a pyrophosphate sequencer (PSQ). Methods of pyrophosphate sequencing are generally described, e.g., in U.S. Pat. Nos. 6,274,320, 6258,568 and 6,210,891, incorporated herein by reference in toto. Briefly, the membranes were soaked for 30 sec in 50 μl of a mixture of ATP sulfurylase and luciferase enzymes (454 Life Sciences). Then, the membrane was placed into a well of the PSQ. The nucleotides were flowed into the PSQ plates in the order of G, A, C, and then T. This was repeated five times. For the sample with no DNA (the negative control), no sequence was detected (FIG. 10A). For the amplified tf2 fragment on the beads on the membrane, the proper sequence was detected (FIG. 10B). Thus, detectable sequence was obtained starting from a small number of DNA template fragments (10 copies per bead), using the amplification and sequencing reactions described herein.

### Example 4

### Methods for Pyrophosphate Sequencing

Any DNA may be sequenced using the procedure described herein. Briefly, beads are filtered to obtain a diameter of 25-30 μm and resuspended at a concentration of 3,500 beads/μl, as described above. Next, 14 μl of the bead solution is placed into a tube for each sample to be sequenced. The beads are pelleted at 13,000 rpm. The supernatant is replaced with 500 μl of a mixture of the three enzymes (6 μl of sulfurylase at 1 mg/ml, 6 μl of luciferase at 3 mg/ml, and 60 μl of Bst polymerase at 50 U/μl) and 428 μl of 1× Assay Buffer containing 1 mg/ml BSA. The tube is placed in a rotator for 1 hr at room temperature, at about one turn every 2 sec. Then, the beads are pelleted by centrifugation at 2,000 rpm for 2.5 min. The beads are washed once with 200 μl of 1× Assay Buffer without BSA. Then the beads are loaded onto a membrane with 30 μm pore for pyrophosphate sequencing.

### Example 5

### Bead Loading Methods

A membrane (e.g., nitrocellulose membrane circle, as described herein) is dipped into bead solution such as 1× Assay Buffer. The membrane is agitated to trap beads in the membrane pores. The membrane/bead mixture is submerged in bead solution. This is stirred or vortexed to trap the beads in the membrane pores. The membrane is used as a bead filter in a sieve, and the bead solution is drained through the membrane using gravity or centrifugal force. On an open-loading Jig and a centered membrane, the bead solution is introduced from the top and drained through the bottom by a pump. Mixing can be used in the cavity of the Jig to ensure uniform bead distribution on the membrane. The loading Jig can include multiple cavities for bead deposits onto different areas of the membrane for different samples or tests. This method can be combined with the loading Jig method as described herein. The beads are loaded using a wicking effect. The membrane is placed on top of other highly hydrophilic membrane or tissue, or other wicking material, and the bead solution is applied to the membrane. Using a pump, the bead solution is flowed across the membrane in an enclosed chamber. The chamber can be placed in any orientation, and the beads can be introduced by various means such as a syringe, pipette, duct, tubing, and the like. The arrayed sample delivery devices (manual or automated) can be used to deposit beads onto discrete regions or patches on the membrane.

### Example 6

### Automated Convective Sequencing Protocol

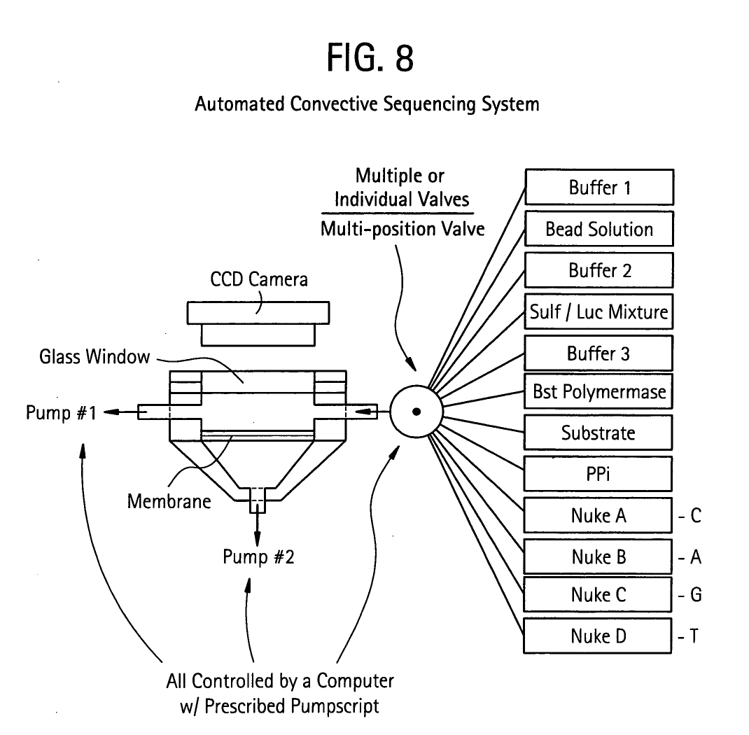

Wash buffers, sample DNA, bead solution, enzyme solutions, and sequencing reagents (substrate, PPᵢ, and nucleotides) are prepared according to the layout of the automated sequencing system (FIG. 8). The sequencing and resistance membranes are incubated in 1× Assay Buffer (AB) containing 1% bovine serum albumin (BSA) for at least 15 min preferably 30 min, to prevent PPᵢ drop during a long sequencing run. The sequence chamber system is assembled with the membranes and connected to pumps and reagents. The beads are loaded with Pump 2 at a flow rate of about 1-2 ml/min depending on the chamber size. The chamber is washed with a wash buffer (1× AB with 1% BSA). Non-binding beads are removed by running Pump 2 while switching to Pump 1. A proper flow rate is set so that it removes loose beads without disrupting the membrane.

A sulfurylase and luciferase mixture is loaded with both Pump 1 and Pump 2 running. This is incubated for 15 min with mixing by the reciprocal movement by Pump 2. The chamber is washed with a wash buffer for polymerase with both pumps running for 5 min. A Bst polymerase (New England Biolabs, Beverly, Mass.) solution is loaded and incubated for 30 min with mixing. The chamber is washed with substrate for 5 min with both pumps running. Alternatively, the Bst polymerase can be mixed with sulfurylase and luciferase for combined infusion with all three enzymes. A 0.1 μM PPᵢ solution is run for signal calibration with a flow rate of 1.5 ml/min for both pumps for 21 sec. After this, Pump 1 is stopped and Pump 2 is continued for another 14 sec. The substrate is washed for 115 sec with Pump 1 at 1 ml/min and Pump 2 at 2 ml/min. The nucleotides are added in order (e.g., C, A, G, T) for a predetermined number of cycles with the same pump procedure as used for PPᵢ, except that a 65 sec substrate wash is used after each nucleotide is added. At the end of the nucleotide runs, PPᵢ is run again to check if enzyme activity changes with time. The data is analyzed using DNA sequencing software.

Additional methods that may be used with the invention can be found in U.S. application Ser. No. 10/191,438 filed Jul. 8, 2002, U.S. application Ser. No. 60/476,592 filed Jun. 6, 2003, U.S. application Ser. No. 60/476,602, filed Jun. 6, 2003, U.S. application Ser. No. 60/476,313 filed Jun. 6, 2003, U.S. application Ser. No. 60/476,504 filed Jun. 6, 2003, and U.S. Pat. No. 6,274,320, which are hereby incorporated by reference herein in their entirety.

The details of one or more embodiments of the invention have been set forth in the accompanying description above. Although any methods and materials similar or equivalent to those described herein can be used in the practice or testing of the present invention, the preferred methods and materials are described above. Other features, objects, and advantages of the invention will be apparent from the description and from the claims.

### REFERENCES

Anderson J R, Chiu D T, Jackman R J, Chemiavskaya O, McDonald J C, Wu H, Whitesides S H, and Whitesides G M. 2000. Fabrication of topologically complex three-dimensional microfluidic systems in PDMS by rapid prototyping. Anal. Chem. 72:3158-64.

Asoh, H, Nishio, K, Nakao, M. Tamamura, T. and Masuda, H. 2001. Conditions for fabrication of ideally ordered anodic porous alumina using pretextured aluminum. J Electrochem. Soc. 148:B152-B156.

Cherukuri S C, Demers R R, Fan Z H H, Levine A W, McBride S E, Zanzucchi P J. 1999. Method and system for inhibiting cross-contamination in fluids of combinatorial chemistry device. U.S. Pat. No. 5,980,704.

Dannoux T, Pujol G, and Root D. 2000. High-density test plate and process of making. U.S. Pat. No. 6,030,829.

Deng T, Wu H, Brittain S T, and Whitesides G M. 2000. Prototyping of masks, masters, and stamps/molds for soft lithography using an office printer and photographic reduction. Anal. Chem. 72:3176-80.

Ehrfeld, W, Hessel, V, and Löwe, H. 2000. Microreactors: New Technology for Modern Chemistry. New York: Wiley-VCH.

Eykamp, W. 1995. Microfiltration and ultrafiltration, pp. 1-43 in Membrane Separation Technology: Principles and Applications, R D Noble and S A Stem, eds. Amsterdam: Elsevier Science BV.

Fumeaux, R C, Rigby, W R, and Davidson, A P. 1989. The formation of controlled porosity membranes from anodically oxidized aluminum. Nature. 337:147-9. Hyman E D. 1988. A new method for sequencing DNA. Anal. Biochem. 174:423-36.

Jansson V and Jansson K. 2002. Enzymatic chemiluminescence assay for inorganic pyrophosphate. Analytical Biochemistry. 304: 135-137.

Jensen N. 2002. Flow-through processing on a microchip for DNA pyrosequencing. Natl. Nanofabrication Users Network. 66-67.

Kane R S, Takayama S, Ostuni E, Ingber D E, and Whitesides G M. 1999. Patterning proteins and cells using soft lithography. Biomaterials. 20:2363-76.

Kulkarni, S S, Funk, E W, and Li, N N. 1992. Ultrafiltration, pp. 391-453 in Membrane Handbook, W S W Ho and K K Sirkar, eds. New York: Van Nostrand Reinhold.

Mardilovich, P P, Govyadinov, A N, Mukhurov, N I, Rzhevskii, A M, and Paterson, R. New and modified anodic alumina membranes. Part I: Treatment of anodic alumina membranes. Part II: Comparison of solubility of amorphous (normal) and polycrystalline anodic alumina membranes. J. Memb. Sci. 98:131-155.

Martin C R. 1994. Nanomaterials—A membrane-based synthetic approach. Science. 266:1961-1966.

Matsuda T and Chung D J. 1994. Microfabricated surface designs for cell culture and diagnosis. ASAIO J. 1:M594-7.

Michael K L, Taylor L C, Schultz S L, and Walt D R. 1998. Randomly ordered addressable high-density optical sensor arrays. Anal. Chem. 70:1242-8.

Nyrén P, Pettersson B, and Uhlén M. 1993. Solid phase DNA minisequencing by an enzymatic luminometric inorganic pyrophosphate detection assay. Anal. Biochem. 208:171-5.

Rai-Choudhury P. 1997. Handbook of Microlithography, Micromachining and Microfabrication. Vol. 1: Microlithography and Vol. 2: Micromachining and Microfabrication. SPIE Press.

Ronaghi M, Uhlén M, and Nyrén P. 1998. A sequencing method based on real-time pyrophosphate. Science. 281:363-5.

Schuller J. 2002. Microfluidics for DNA pyrosequencing. Natl. Nanofabrication Users Network. 76-77.

Taylor L C and Walt D R. 2000. Application of high-density optical microwell arrays in a live-cell biosensing system. Anal. Biochem. 278:132-42

Zhu H, Klemic J F, Chang S, Bertone P, Casamayor A, Klemic K G, Smith D, Gerstein M, Reed M A, and & Snyder M. 2000. Analysis of yeast protein kinases using protein chips. Nature Genet. 26:283-89.

What is claimed is:

1. A membrane reactor comprising:
a. a planar array layer comprising a top surface and bottom surface, wherein the top surface comprises a plurality of wells with beads disposed in a plurality of the wells, wherein the top surface further comprises sidewalls around a periphery of the wells and bases at the bottom surface of the wells,
wherein there is a maximum of one bead per well, wherein the sidewalls of the well extend higher than the bead in the well, wherein the sidewalls and bases of the wells comprise one or more opaque materials, and wherein the bases of the wells are substantially permeable to an aqueous solution;
b. a porous high flow resistance membrane layer comprising a top and bottom surface, wherein the top surface of the membrane layer is in contact with the bottom surface of the planar array layer, wherein pores of the membrane layer are permeable to the aqueous solution but impermeable to the beads in the wells, and wherein flow resistance of the membrane layer for the aqueous solution is at least 10-fold greater than flow resistance of the planar array layer;
c. optionally, a permeable structural support layer which has reduced flow resistance for the aqueous solution as compared to the planar array layer and the porous high flow resistance membrane layer; and
d. a fluid flow of aqueous solution passing through layers of the membrane reactor, wherein the fluid flow is substantially perpendicular to the top surface of the planar array layer, and wherein the fluid flow retains the beads in the wells in the planar array layer.

2. The membrane reactor of claim 1, wherein the planar array layer provides a spacing of wells of less than 100 μm center to center.

3. The membrane reactor of claim 1, wherein the planar array layer provides a spacing of wells of about 5 μm to 200 μm center to center.

4. The membrane reactor of claim 1, wherein the planar array layer comprises wells with a well width of about 15 μm to 100 μm.

5. The membrane reactor of claim 1, wherein the planar array layer comprises wells with a well width of about 20 to 35 μm.

6. The membrane reactor of claim 1, wherein the planar array layer comprises wells having one or more shapes selected from the group consisting of substantially round, square, oval, rectangular, hexagonal, crescent, and star shapes.

7. The membrane reactor of claim 1, wherein the planar array layer comprises at least 10,000, 50,000, 100,000, or 250,000 wells.

8. The membrane reactor of claim 1, wherein the planar array layer comprises at least 100, 100-1000, 1000-10,000, 10,000-20,000, 20,000-30,000, or 32,000 wells per mm².

9. The membrane reactor of claim 1, wherein the high flow resistance membrane layer has a pore size of about 0.2 to 12 μm in diameter.

10. The membrane reactor of claim 1, wherein the high flow resistance membrane layer has a pore size of about 0.5 to 12 μm in diameter.

11. The membrane reactor of claim 1, wherein the high flow resistance membrane layer has a pore size of about 0.1 to 5 μm in diameter.

12. The membrane reactor of claim 1, wherein the high flow resistance membrane layer has a thickness of about 10 to 23 μm, about 9 to 23 μm, or about 10 to 20 μm.

13. The membrane reactor of claim 1, wherein the high flow resistance membrane layer has a flow resistance for the aqueous solution that is 100-fold greater than the flow resistance of the planar array layer.

14. The membrane reactor of claim 1, wherein the high flow resistance membrane layer has a pore diameter that is less than 10% of a pore diameter of the planar array layer.

15. The membrane reactor of claim 1, wherein the high flow resistance membrane layer has a pore diameter that is less than 1% of a pore diameter of the planar array layer.

16. The membrane reactor of claim 1, wherein the planar array layer comprises one or more metals or metal-plated materials.

17. The membrane of claim 1, wherein the high flow resistance membrane layer comprises one or more materials selected from the group consisting of glass, silicon, polyester, and polycarbonate materials.

18. The membrane reactor of claim 1, wherein the structural support layer comprises one or more materials selected from the group consisting of metal, ceramic, and silicon.

19. The membrane reactor of claim 1, wherein the sidewalls of the wells of the planar array layer comprise one or more reflective materials.

20. The membrane reactor of claim 1, wherein the bases of the wells of the planar array layer comprise one or more reflective materials.

21. The membrane reactor of claim 1, further comprising at least one DNA molecule immobilized on the planar array layer or on the bead.

22. The membrane reactor of claim 1, further comprising at least one sequencing enzyme immobilized on the planar array layer or on the bead.

23. The membrane reactor of claim 22, wherein the sequencing enzyme is selected from the group consisting of sulfurylase, luciferase, polymerase, hypoxanthine phosphoribosyltransferase, xanthine oxidase, uricase, apyrase, and peroxidase.

24. A method of identifying a base at a target position in a sample DNA sequence comprising:
a. providing the membrane reactor of claim 1, wherein the plurality of wells comprise at least one sample DNA immobilized on the bead;
b. providing a sequencing enzyme and at least one extension primer that hybridizes to the sample DNA immediately adjacent to a target on the sample DNA;
c. adding different deoxynucleotides or dideoxynucleotides successively to the sample DNA and extension primer, whereby the deoxynucleotide or dideoxynucleotide will only become incorporated and release pyrophosphate (PPᵢ) if it is complementary to the base in the target position of the sample DNA; and
d. detecting any release of PPᵢ to determine which deoxynucleotide or dideoxynucleotide is incorporated, thereby identifying a base that is complementary to the base at the target position.

25. The method of claim 24, wherein the different deoxynucleotides or dideoxynucleotides are added to the DNA sequence by a fluid flow of aqueous solution that is substantially perpendicular to the top surface of the planar array layer.

26. The method of claim 25, wherein the fluid flow has a flow rate of about 0.15 ml/minute/cm² to 4 ml/minute/cm².

27. The method of claim 24, wherein the sequencing enzyme is immobilized on one or more beads.

28. The method of claim 27, wherein the sequencing enzyme is selected from the group consisting of sulfurylase, luciferase, polymerase, hypoxanthine phosphoribosyltransferase, xanthine oxidase, uricase, apyrase, and peroxidase.

29. A microimaging lens system for analyzing the membrane reactor of claim 1 comprising:
a. a front lens group for collecting a light emission from a sequencing reaction; and
b. a rear lens group for imaging the light emission in a spatially ordered manner onto an optical detector.

30. The microimaging lens system of claim 29, wherein at least one the lens group has a focal length selected from at least 30 mm, at least 50 mm, and at least 70 mm.

31. The microimaging lens system of claim 29, wherein at least one the lens group has an aperture brighter than or equal to 4.0 or 2.8.

32. The microimaging lens system of claim 29, wherein at least one the lens group has a numeric aperture larger than or equal to 0.1, 0.2, or 0.3.

33. The microimaging lens system of claim 29, wherein the front lens group and rear lens group are identical.

34. The microimaging lens system of claim 29, further comprising a solid state optical detector.

35. The microimaging lens system of claim 34, wherein the solid state optical detector is a CCD array.

36. The microimaging lens system of claim 29, further comprising a structure to reduce or prevent background light from reaching the optical detector.

37. The microimaging lens system of claim 36, wherein the structure is an opaque tube comprising a first end which forms a light tight fit to one end of the microimaging lens and a second end which forms a light tight fit with the optical detector.

38. The microimaging lens system of claim 36, wherein the structure is an opaque tube comprising a first end which forms a light tight fit to one end of the microimaging lens and comprising a second end which forms a light tight fit to the membrane reactor.

39. A sequencing cartridge comprising:
a. a flow chamber enclosing the membrane reactor of claim 1;
b. an optical window proximal to top surface of the planar array layer for the optical examination of the membrane reactor;
c. an inlet port tangential to top surface of the planar array layer for delivering sequencing reagents;
d. a first outlet port tangential to top surface of the planar array layer for removal of sequencing reagents; and
e. a second outlet port perpendicular to bottom surface of the membrane layer for removal of effluents.

40. The sequencing cartridge of claim 39, wherein the optical window is circular.

42. The sequencing cartridge of claim 39, wherein the flow chamber is substantially funnel shaped with the wide section of the funnel proximal to the planar array layer and the narrow section of the funnel proximal to the second outlet port.

43. The sequencing cartridge of claim 39, wherein the support structure comprises a porous solid surface.

44. The sequencing cartridge of claim 39, wherein the inlet port is in fluid communication with a pump for controlling a flow of fluids through the inlet port.

45. The sequencing cartridge of claim 39, wherein the first outlet port is in fluid communication with a first pump for controlling a flow of fluids through the first outlet port.

46. The sequencing cartridge of claim 39, wherein the second outlet port is in fluid communication with a second pump for controlling a flow of fluids through the second outlet port.

47. The sequencing cartridge of claim 39, wherein the inlet port and the first and the second outlet port is each in fluid communication with a different pump for controlling a flow of fluids so that a flow of fluids perpendicular to the membrane reactor and a flow of fluid tangential to the membrane reactor can be controlled simultaneously.


SEQUENCE LISTING (11 sequences)

SEQ ID NO: 1 - DNA, 20 bp, Artificial
  Synthetic Primer
caattaaccctcactaaagg

SEQ ID NO: 2 - DNA, 21 bp, Artificial
  Synthetic Primer
gtaatacgactcactataggg

SEQ ID NO: 3 - DNA, 136 bp, Artificial
  Synthetic Primer
cgatcaagcgtacgcacgtggttgttaaagcttttttgaaagttaatctcctggttcaccgtctgctcgtatgcggttaccaggtcggcggccgccacgtgtgcgcgcgcgggactaatcccggttcgcgcgtcgg

SEQ ID NO: 4 - DNA, 20 bp, Artificial
  Synthetic Probe
caattaaccctcactaaagg

SEQ ID NO: 5 - DNA, 88 bp, Artificial
  Synthetic DNA Sequence
atgccgcaaaaacgcaaaacgcaaacgcaacgcatacctctccgcgtaggcgctcgttggtccagcagaggcggccgcccttgcgcga

SEQ ID NO: 6 - DNA, 20 bp, Artificial
  Synthetic Primer
ccatctgttccctccctgtc

SEQ ID NO: 7 - DNA, 129 bp, Artificial
  Synthetic DNA Template
atgccgcaaaaacgcaaaacgcaaacgcaacgcatacctctccgcgtaggcgctcgttggtccagcagaggcggccgcccttgcgcgagcagaatggcggtagggggtctagctgcgtctcgtccgggg

SEQ ID NO: 8 - DNA, 20 bp, Artificial
  Synthetic Primer
ccatctgttgcgtgcgtgct

SEQ ID NO: 9 - DNA, 20 bp, Artificial
  Synthetic Primer
cgtttcccctgtgtgccttg

SEQ ID NO: 10 - DNA, 84 bp, Artificial
  Synthetic DNA Sequence
cagtcagtcagtcagtcagtcagtcagtcagtcagtcagtcagtcagtcagtcagtcagtcagtcagtcagtcagtcagtcagt

SEQ ID NO: 11 - DNA, 20 bp, Artificial
  Synthetic Amplified DNA
gatcgatcgatcgatcgatc